(19) United States
(12) Patent Application Publication
(10) Pub. No.: US 2009/0304589 A1
(43) Pub. Date: Dec. 10, 2009

(54) RADIATION SENSITIVE LIPOSOMES

(75) Inventors: Bruce Bondurant, Durham, NC (US); Kathy A. McGovern, Tucson, AZ (US); Robert M. Sutherland, Menlo Park, CA (US)

Correspondence Address: Townsend and Townsend and Crew LLP (Varian), Two Embarcadero Center, Eighth Floor, San Francisco, CA, 94111, US

(73) Assignee: Varian Medical Systems,Inc., Palo Alto, CA (US)

(21) Appl. No.: 12/238,934

(22) Filed: Sep. 26, 2008

Related U.S. Application Data
(60) Provisional application No. 60/976,309, filed on Sep. 28, 2007.

(51) Int. Cl. A61K 49/00; A61K 9/127; A61P 43/00

(52) U.S. Cl. 424/9.1; 424/450

(57) ABSTRACT

The present invention relates to a radiation sensitive liposome, and the use of this liposome as carrier for therapeutic and diagnostic agent(s). In particular, the invention encompasses a liposome composition comprising a stable liposome-forming lipid and a polymerizable colipid, and a chain transfer agent. The present invention further contemplates methods of diagnosing and treating conditions and diseases that are responsive to liposome-encapsulated or associated agents.

### CROSS-REFERENCES TO RELATED APPLICATIONS

The present application claims priority to U.S. Ser. No. 60/976,309, filed Sep. 28, 2007, herein incorporated by reference in its entirety.

### STATEMENT AS TO RIGHTS TO INVENTIONS MADE UNDER FEDERALLY SPONSORED RESEARCH OR DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK

Not Applicable

### BACKGROUND OF THE INVENTION

a) Field of the Invention

The present invention relates to liposomes comprising chain transfer agents or redox initiators. The invention further relates to the use of these liposomes as carriers for therapeutic and diagnostic agents.

b) Description of Related Art

Liposomes are microscopic vesicles consisting of concentric lipid bilayers. Structurally, liposomes range in size and shape from long tubes to spheres, with dimensions from a few hundred Angstroms to fractions of a millimeter. Regardless of the overall shape, the bilayers are generally organized as closed concentric lamellae, with an aqueous layer separating each lamella from its neighbor. Vesicle size normally falls in a range of between about 20 and about 30,000 nm in diameter. The liquid film between lamellae is usually between about 3 and 10 nm. A variety of methods for preparing liposomes have been described in the literature. For specific reviews and information on liposome formulations, reference is made to reviews by Pagano and Weinstein (Ann. Rev. Biophys. Bioeng. 1978, 7:435-68) and Szoka and Papahadjopoulos (Ann. Rev. Biophys. Bioeng. 1980, 9:467-508) and additionally to U.S. Pat. Nos. 4,229,360; 4,224,179; 4,241,046; 4,078,052; and 4,235,871, the disclosures of which are incorporated by reference herein.

Biological cell membranes exploit the amphiphilic nature of lipids to create anatomical boundaries, e.g., the plasma membrane and the mitochrondrial membrane. During the early 1960s researchers demonstrated that certain classes of lipids, especially glycerophospholipids, could be used to form protein-free model membranes. They developed methods for the preparation of supported bilayer lipid membranes (BLM) and discovered that dried thin films of phospholipids spontaneously hydrate to yield self-supported closed bilayer assemblies of on the order of a hundred thousand lipid molecules, i.e., liposomes. The lipid bilayer in each model membrane is a two-dimensional fluid composed of lipids with their hydrophilic head groups exposed to water and their hydrophobic tails aggregated to exclude water. The bilayer structure is highly ordered, yet dynamic due to the rapid lateral motion of the lipids within the plane of each half of the bilayer.

Typically, liposomes can be divided into three categories based on their overall size and the nature of the lamellar structure. The three classifications, as developed by the New York Academy Sciences Meeting, “Liposomes and Their Use in Biology and Medicine,” of December, 1977, are multi-lamellar vesicles (MLV's), small uni-lamellar vesicles (SUV's) and large uni-lamellar vesicles (LUV's). SUV's range in diameter from approximately 20 to 50 nm and consist of a single lipid bilayer surrounding an aqueous compartment. Unilamellar vesicles can also be prepared in sizes from about 50 nm to 600 nm in diameter. While unilamellar vesicles are of fairly uniform size, MLV's vary greatly in size up to 10,000 nm, or thereabouts, are multi-compartmental and contain more than one bilayer. LUV liposomes are so named because of their large diameters which range from about 600 nm to 30,000 nm; they can contain more than one bilayer.

Liposomes may be prepared by a number of methods, not all of which can produce the three different types of liposomes. For example, ultrasonic dispersion by means of immersing a metal probe directly into a suspension of MLV's is a common way for preparing SUV's. Preparing liposomes of the MLV class usually involves dissolving the lipids in an appropriate organic solvent and then removing the solvent under a gas or air stream. This leaves behind a thin film of dry lipid on the surface of the container. An aqueous solution is then introduced into the container with shaking in order to free lipid material from the sides of the container. This process disperses the lipid, causing it to form into lipid aggregates or liposomes. Liposomes of the LUV variety may be made by slow hydration of a thin layer of lipid with distilled water or an aqueous solution. Alternatively, liposomes may be prepared by lyophilization. This process comprises drying a solution of lipids to a film under a stream of nitrogen. This film is then dissolved in a volatile, freezable, organic solvent, e.g., cyclohexane or t-butanol, frozen, and placed on a lyophilization apparatus to remove the solvent. To prepare a pharmaceutical formulation containing a water-soluble drug, an aqueous solution of the drug is added to the lyophilized lipids, whereupon liposomes are formed.

Lipophilic drugs may be incorporated into the bilayer of the liposome by dissolving them with the lipid in the organic phase and then removing the organic phase. Hydration with the aqueous phase will result in the incorporation of the lipophilic drug into the liposomal bilayer structure. This applies both to lyophilization and thin film methods. The encapsulation characteristics and biocompatibility of liposomes make them ideal carriers for therapeutic agents. Research efforts have been devoted to the development of liposomes for the delivery of drugs in the body. Successful in vitro studies have led to clinical trials of liposome-encapsulated amphotericin B, anthracyclines, and other drugs.

Suitably designed liposomes can extend the circulation time and target the drug to particular tissues of the body (Allen, T. M., Liposome Res. 1992, 2:289-305; Allen, T. M., Trends Pharm. Sci. 1994, 15:215-220; Blume et al., Biochim. Biophys. Acta 1993, 1149:180-184; Klibanov et al., J Liposome Res. 1992, 2:321-334; Lasic et al., D. Science 1995, 267:1275-1276; Lasic et al., Stealth Liposomes, CRC Press: Boca Raton, Fla., 1995). The delivery of liposomes to the desired sites depends in part on long circulation times in the body, which can only be accomplished by reducing the uptake of liposomes by the mononuclear phagocytic system (MPS). In recent years several means have been described to sterically stabilize liposomes in order to increase their period of circulation (Lasic et al., Stealth Liposomes, supra; Woodle et al., Biochim. Biophys. Acta 1992, 1113:171-199). A frequently used method is the attachment of polyethylene glycol (PEG) to some of the lipids in the liposome. This is usually accomplished by the chemical reaction of PEG or its derivatives with the amino function group of phosphatidyl ethanolamines (PE), e.g., methyl-PEG coupled to PE via a carbamyl linkage (Allen et al., Biochim. Biophys. Acta 1991, 1066:29-37); activation of methoxy-PEG with cyanuric acid (Klibanov et al., FEBS Lett. 1990, 268:235-243; Mori et al., FEBS Lett. 1991, 284:263-271); and conjugation of PEG to PE with succinimidyl succinate (Klibanov et al., FEBS Lett., supra; Mori et al., FEBS Lett., supra; Woodle et al. Proceed. Intern. Symp. Control. Rel. Bioact. Mater 1990, 17:77).

In addition to extended circulation times, the successful delivery of liposomes to specific tissue sites requires the liposomes to enter the interstitium. Tumors represent a specific tissue site of considerable therapeutic interest. Several research groups have reported the increased localization of sterically stabilized liposomes (PEG-liposomes) at tumor sites. The increased permeability of the vasculature at tumor sites (due to angiogenic factors secreted by tumors) allows liposomes to escape the capillaries to reach the tumor interstitial space. Sterically stabilized liposomes are more likely to accumulate at these sites because of their sustained concentration in the blood. Furthermore, it is known that the hydrophilic surface polymer may facilitate the transit from the capillaries to the tumor site. Reports of passive targeting of PEG-liposomes to tumors, including murine colon carcinomas, murine lymphomas, murine mammary carcinomas, human squamous cell lung carcinomas in SCID mice are known in the art.

Specific targeting via antibodies coupled to liposomes has been observed as well. Monoclonal antibody (mAb) conjugated sterically stabilized liposomes are known to localize at squamous cell carcinomas of the lung in mice and effectively deliver doxorubicin to these sites. Although the coupling of mAbs to conventional liposomes appears to increase their rate of clearance from the blood stream, the mAb conjugated PEG-liposomes remain in circulation long enough to accumulate at their target cells.

In order for the liposomes to reach the target site without significant loss of their contents, passive leakage must be slow relative to the time required for liposomes to circulate and escape the vasculature. However, it has been shown that once sterically stabilized liposomes have accumulated at tumor sites the slow passive leakage of encapsulated chemotherapeutics, e.g., doxorubicin, can significantly affect the cells at that site. It would be desirable to stimulate enhanced release of the encapsulated agent(s) from the liposomes once the liposomes are at the target site.

The delivery of liposomes to desired anatomical sites depends in part on long liposome circulation times, which can be achieved if the liposomes are sterically stabilized by tethered hydrophilic polymers, such as PEG-PE. The increased permeability of the vasculature at tumor sites allows PEG-liposomes to escape the capillaries to reach the tumor interstitial space. However, once the PEG-liposomes are at the tumor site the PEG groups can interfere with rapid release of the encapsulated reagents. Consequently, it is a continuing challenge to find methods to trigger the release of reagents from PEG-liposomes.

Ideally, such a stimulus would be spatially and temporally selective, in a manner analogous to photodynamic therapy. In photodynamic therapy, certain porphyrins and other photosensitizers are administered systemically, absorbed by cells, and upon exposure to visible light focused at the target site. Hence, the photodynamic effect results in the localized destruction of the target cells. This effect has proven useful for the treatment of cancer cells in areas of the body that are accessible to coherent light via fiber optics. In principle, the successful use of light (or other forms of radiation) to treat disease can be broadened to include a wide variety of therapeutic agents, particularly, if light is used to release the agent.

Several strategies have been employed to design photosensitive liposomes. These include the photochemical modification of individual lipids in the bilayer, i.e., lipid photochemistry; the photo induced change in the association of polyelectrolytes with liposomes; and the photoinitiated polymerization of some or all of the lipids in the liposome, i.e., photopolymerization. A characteristic of photopolymerization processes is their multiplicative nature, which generally results in a greater perturbation of the bilayer membrane for equivalent light exposures. An extensive review of methods to photochemically reorganize lipid bilayers has been published (O'Brien et al., Bioorganic Photochemistry 1993, 2:111-167).

The photopolymerization of selected lipids in a multicomponent membrane can alter the lateral distribution of lipids within the bilayer to form domains enriched in polymerized lipids (Armitage et al., Adv. Polym. Sci. 1996, 126:53-85). It is known that processes that cause the phase separation of PE and other lipids can trigger lamellar to nonlamellar phase transition(s). The polymerization of two or multi-component lipid bilayers, with one polymerizable and other nonpolymerizable component(s), can cause lipid domain formation. The polymerizable lipids form covalently linked domains as the reaction proceeds, which in turn produces domains of the nonpolymerizable component(s). O'Brien and coworkers showed that if the nonpolymerizable component prefers a nonlamellar rather than a lamellar structure the membrane will be destabilized (Lamparski et al., Biochemistry 1992, 31:685-694; Bennett et al., Biochemistry 1995, 34:3102-3113). Phosphatidyl ethanolamines are of particular interest because they form nonbilayer structures under physiological conditions. The polymerizable phosphatidylcholines (PC) and phosphatidyl ethanolamines (PE) form stable two-component liposomes and are stable prior to polymerization, but are destabilized by photopolymerization of a bis-SorbPC which contains a photosensitive sorbyl moiety at the terminal end of each acyl chain. Consequently, the photopolymerization of properly designed lipid bilayers can initiate the localized destabilization of the bilayer, which is observed either as the leakage of encapsulated reagents (Lamparski et al., supra) or the fusion of bilayer liposomes (Bennett et al., supra).

A major strategy for the formation of polymerized bilayers and other supramolecular assemblies is the preparation of polymerizable lipid monomers, the formation of the lipid assembly such as bilayer membranes from the monomer, and the subsequent chain polymerization of the monomers in the assembly. Polymerizable lipids have been prepared by introduction of the reactive group into different regions of the lipid molecule. A schematic representation of these types of polymerizable lipids is shown in FIG. 1. As shown in FIG. 1, polymerization strategies A and B have no direct influence on the membrane-water interface. The mobility of the lipid chains is significantly decreased by polymerization in these systems. In contrast strategies C and D alter the membrane-water interface, but have less effect on the hydrophobic interior of the membrane. The polymerizable lipids as shown in FIG. 1, with only one reactive group per lipid, form linear polymer chains in supramolecular assemblies. The presence of a second polymerizable group per molecule (not shown) allows crosslinking of the polymer chains.

A host of reactive moieties have been utilized to modify the above lipids to make them polymerizable. These groups include but not limited to diacetylene, acryloyl, methacryloyl, itaconyl, dienoyl, sorbyl, muconyl, styryl, vinyl, thiol (or lipoyl), and chain terminal isocyanates. Systematic studies of the relationship between polymer chain length, i.e., degree of polymerization (Xₙ), and the molar ratio of monomer to initiator ([M]/[I]) revealed that Xₙ was proportional to [M]/[I]. Moreover, these studies showed that the relative reactivity of monomers in bilayer membranes is similar to values obtained from the multitude of solution polymerization studies. Consequently, an acryloyl lipid monomer in a bilayer is four to five times more reactive than a diene containing lipid monomer.

Polymerization of lipids in bilayer membranes can be initiated by various methods, including photo, thermal, ionizing and redox initiation. Diacetylenic, butadienic, vinylic, acryloylic, methacryloylic, and thiolic units have been used as polymerizable units in acyl chains.

The methods described above relating to radiant energy initiated polymerization of the lipid bilayer rely on ultraviolet light. The potential utility of polymerizable liposomes for drug delivery, diagnostics, and reagent release is limited if only ultraviolet light can be used for initiation of polymerization. UV light can only be used where the target tissue is superficially accessible to the light source. Liposomes that exist at deeper tissue levels would not be accessible to UV light and liposome-encapsulated or associated diagnostic or therapeutic agents could therefore not be released. Hence, a better system is required to achieve destabilization of liposomes.

International Patent Application No. WO 01/39744 and U.S. Pat. No. 6,989,153 describe a liposome delivery system comprising lipids and ionizing radiation polymerizable colipids. If the liposome delivery system is polymerized with ionizing radiation under conditions wherein the lipids and colipids are in pre-existing discrete domains, the liposomes are significantly destabilized resulting in release of the diagnostic or therapeutic agent as compared to ionizing radiation of liposomes not in pre-existing discrete domains.

There is a need for a liposomal delivery system which is (1) stable upon delivery to a patient such that there is no or little passive leakage of the releasable agent into the patient; (2) able to circulate for a sufficient period in the patient to reach the target site and (3) able to release the therapeutic or diagnostic agent quickly upon ionizing radiation.

### BRIEF SUMMARY OF THE INVENTION

The present invention relates to liposome delivery system(s) or liposome(s) that is/are radiation sensitive and methods of producing them. The present invention further encompasses methods of diagnosing and treating conditions and diseases that are responsive to liposome-encapsulated or associated agents.

One embodiment of the present invention provides a polymerizable liposome, comprising a stable liposome-forming lipid, an ionizing radiation polymerizable colipid, a chain transfer agent and a releaseable agent. More specifically, the radiation sensitive liposome of the instant invention comprises a radiation polymerizable colipid(s) in the liposomal membrane which forms discrete domains in the liposome at the body temperature of a patient and polymerizes when exposed to ionizing radiation, upon which the liposomal membrane destabilizes and allows leakage of the releaseable agent. The chain transfer agent transfers the free radical ions generated by the radiation into the bilayer to increase the polymerization of the radiation polymerizable colipid.

In one embodiment of the invention, the chain transfer agent has a cleavable hydrophilic group which is cleaved upon exposure to radiation. In another embodiment, the chain transfer agent is located near the liposome surface.

In one embodiment of the invention, the chain transfer agent is a thiol chain transfer agent. Further in another embodiment of the invention, the thiol chain transfer agent is an unsaturated thiol. Further in another embodiment of the invention, the thiol chain transfer agent is a branched thiol. Examples of thiol chain transfer agents include but are not limited to: DDM (dodecyl mercaptan), (4E,6E,11E,13E)-pentadeca-4,6,11,13-tetraene-1-thiol, (10E,12E)-4,7-dimethylenetetradeca-10,12-diene-1-thiol.

In yet another embodiment of the invention, the chain transfer agent is an amphiphilic non-thiol reagent. Examples of amphiphilic non-thiol chain transfer agents include but are not limited to: halocarbons such as carbon tetrachloride, tertiary amines such as N,N-dimethyldodecyl amine and 2-hexadecanone.

In another embodiment of the invention, the chain transfer agent is selected from the group consisting of: CH₃(CH₂)ₙSH, wherein n is 7 to 13; HSCH₂CH═C(CH₃)[(CH₂)₃CH(CH₃)]ₚCH₃, wherein p is 1 to 3. In yet another embodiment of the invention, the chain transfer agent is a Barton ester, which includes but not limited to:

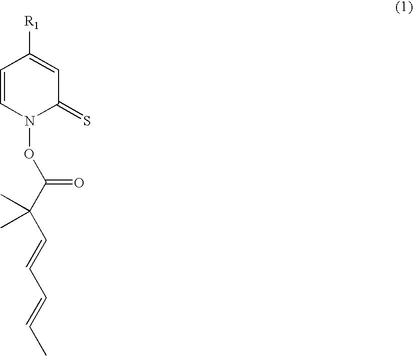

wherein R₁ is a hydrophilic group selected from —(CH₂)ₚCOOH, p is 1 to 4; —(CH₂)ₚN+(CH₃)₃, p is 1 to 4; or —CH₂CH₂O)_{w}COOH, w is 3 to 20.

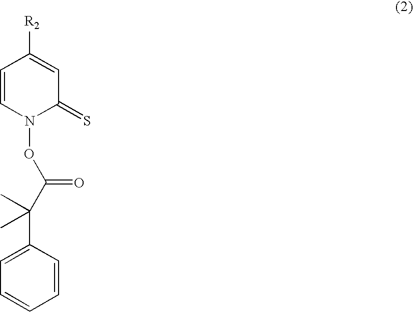

wherein R₂ is a hydrophilic group selected from —CH₂)ₚCOOH, p is 1 to 4; —CH₂)ₚN+(CH₃)₃, p is 1 to 4; or —(CH₂CH₂O)_{w}CH₃, w is 3 to 20.

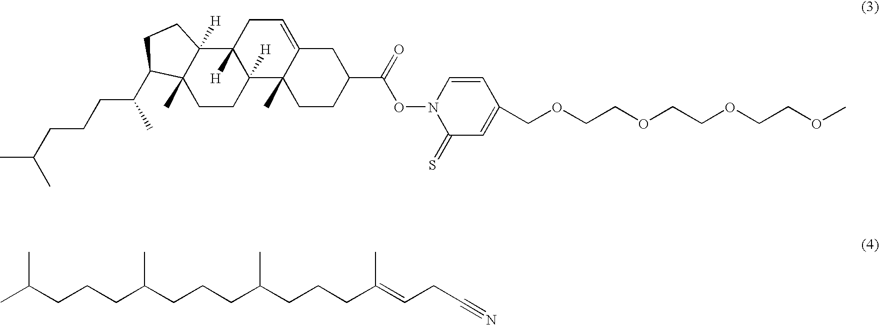

In another embodiment of the invention, the chain transfer agent is a thiol cholesterol. Examples include but are not limited to:

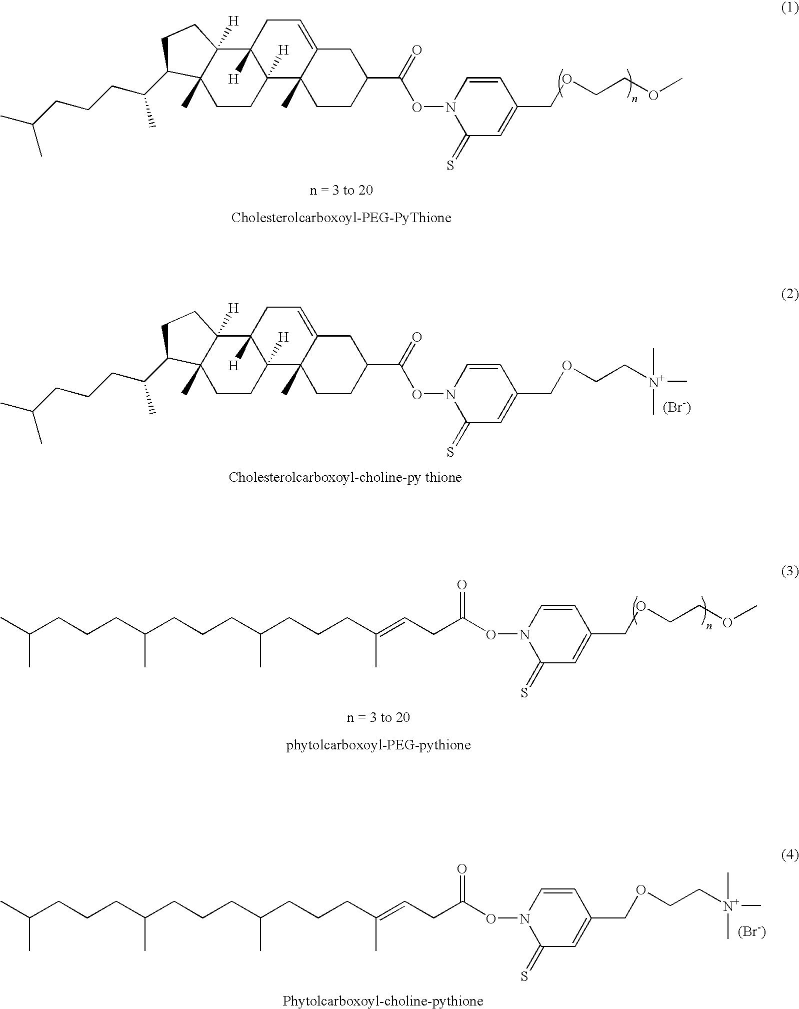

In yet another embodiment, the invention provides a radiation polymerizable liposome comprising a stable liposome-forming lipid, a radiation polymerizable colipid and a biologically acceptable oxidizing agent. The oxidizing agent is selected from the group include are but not limited to: potassium bromate, potassium persulfate (K₂S₂O₈) (or other bio-compatible persulfates), iron(III) sulfate (Fe(SO₄)₃) (or other bio-compatible Fe(III) salts), hydrogen peroxide (H₂O₂), tert-butyl hydroperoxide ((CH₃)₃COOH), and peroxy-carboxylic acids such as CH₃C(═O)OOH.

In another embodiment, the liposome further comprises a steric stabilizer. Preferably the steric stabilizer is polyethylene glycol functionally attached to a lipid.

Another embodiment further contemplates a liposome that can be targeted to a tumor site through attachment of at least one peptide to the liposome. Peptides that target liposome(s) to tumor sites include, but are not limited to, peptide sequences, peptide fragments, antibodies, antibody fragments, and antigens.

The lipid is selected from the group consisting of phosphatidylcholine (PC), phosphatidylethanolamine (PE), phosphatidic acid (PA), phosphatidylglycerol (PG), nonmatural lipid(s), and cationic lipid(s).

The radiation polymerizable colipid is selected from the group consisting of mono-lipids with polymerizable moieties, bis-lipids with polymerizable moieties, and mixtures of mono- and bis-lipids with polymerizable moieties. More specifically, polymerizable colipid(s) include, but are not limited to, mono-, bis-, and heterobifunctional, diacetylenyl, acryloyl, methacryloyl, dienoyl, dienyl, sorbyl, muconyl, styryl, vinyl, and lipoyl colipid(s).

Releasable agent(s) are liposome-encapsulated or associated molecules. Such encapsulated molecules may be water soluble molecules. Alternatively, liposome(s) may comprise lipid associated molecules. The releasable agent(s) include, but are not limited to, therapeutic agents and diagnostic agents. Examples of therapeutic agents include, but are not limited to, chemotherapeutics, biological response modifiers, biological cofactors, pharmaceuticals and radiopharmaceutical, cell toxins, radiation sensitizers, and genetic materials. Examples of diagnostic agents include, but are not limited to, contrast agents, iodinated agents, radiopharmaceutical, fluorescent compounds and fluorescent compounds coencapsulated with a quencher, agents containing MRS/MRI sensitive nuclides, and genetic material encoding contrast agents. Carrying a therapeutic or diagnostic agent within or associated with a liposome provides for a biocompatible and non-toxic means of in vivo delivery. The liposome(s) can be formulated to include a variety of compositions and structures that are potentially non-toxic, degradable, and nonimmunogenic.

In yet another embodiment, the invention provides a liposome composition comprising a mixture of a first liposome and a second liposome. The first liposome comprises a stable liposome-forming lipid, a radiation polymerizable colipid and a biologically acceptable oxidizing reagent. The second liposome comprises a stable liposome-forming lipid, a radiation polymerizable colipid and a releaseable agent. The second liposome may further comprise a chain transfer agent.

The present invention also encompasses a pharmaceutical composition comprising a liposome of the present invention. The pharmaceutical composition may include or be associated with an additional suitable pharmaceutical carrier or diluent.

In one embodiment the pharmaceutical composition comprises (1) a liposome comprising a stable liposome forming lipid, a radiation polymerizabel colipid, a chain transfer agent and a releaseable agent and (2) a pharmaceutically acceptable carrier.

In another embodiment the pharmaceutical composition comprises (1) a liposome comprising a stable liposome forming lipid, a radiation polymerizable colipid, a biologically acceptable oxidizing agent, and (2) a pharmaceutically acceptable carrier.

In another embodiment, the pharmaceutical composition comprises (1) a liposome comprising a stable liposome forming lipid, a radiation polymerizable colipid, a biologically acceptable oxidizing agent, (2) a liposome comprising a stable liposome forming lipid, a radiation polymerizabel colipid, and a releaseable agent and (3) a pharmaceutically acceptable carrier.

In another embodiment, the pharmaceutical composition comprises (1) a liposome comprising a stable liposome forming lipid, a radiation polymerizable colipid, a biologically acceptable oxidizing agent, (2) a liposome comprising a stable liposome forming lipid, a radiation polymerizabel colipid, a chain transfer agent, and a releaseable agent and (3) a pharmaceutically acceptable carrier.

In another embodiment, the instant invention provides for a method of treating a condition responsive to a therapeutic agent, comprising the steps of (i) administering to a patient a pharmaceutical composition comprising a liposome comprising a stable liposome forming lipid, a radiation polymerizable colipid, a chain transfer agent, and a releaseable agent and a pharmaceutically acceptable carrier or diluent; and (ii) subjecting the patient to radiation in order to destabilize the liposome and release the therapeutic agent encapsulated in or associated with the liposome.

In another embodiment, the instant invention provides for a method of treating a condition responsive to a therapeutic agent, comprising the steps of (i) administering to a patient a pharmaceutical composition comprising (1) a liposome comprising a stable liposome forming lipid, a radiation polymerizable colipid, a biologically acceptable oxidizing agent, (2) a liposome comprising a stable liposome forming lipid, a radiation polymerizabel colipid, a chain transfer agent, and a therapeutic agent and (3) a pharmaceutically acceptable carrier and subjecting the patient to radiation in order to destabilize the liposome and release the therapeutic agent encapsulated in or associated with the liposome.

In one embodiment, the radiation dosage ranges from about 50 to about 2000 rads.

In another embodiment, the radiation dosage ranges from about 100 to about 3000 rads. In another embodiment the dosage ranges from about 500 to about 2500 rads. Examples of therapeutic agents include, but are not limited to, chemotherapeutics, biological response modifiers, biological cofactors, pharmaceuticals and radiopharmaceuticals, cell toxins, radiation sensitizers, and genetic materials. Examples of conditions that are responsive to liposome-encapsulated or associated therapeutic agent(s) include, but are not limited to, cancer, immune disorders, developmental disorders, and genetic disorders.

In still another embodiment, the instant invention provides for a method of diagnosing the presence or progression of a disease, comprising the steps of (i) administering to a patient a pharmaceutical composition comprising a liposome comprising a stable liposome forming lipid, a radiation polymerizable colipid, a chain transfer agent, and a diagnostic agent and a pharmaceutically acceptable carrier or diluent; (ii) subjecting the patient to radiation in order to destabilize the liposome and release the diagnostic agent encapsulated in or associated with the liposome, and (iii) diagnosing the disease through use of molecular imaging techniques.

In another embodiment, the instant invention provides for a method of diagnosing the presence or progression of a disease, comprising the steps of (i) administering to a patient a pharmaceutical composition comprising (1) a liposome comprising a stable liposome forming lipid, a radiation polymerizable colipid, a biologically acceptable oxidizing agent, (2) a liposome comprising a stable lipo some forming lipid, a radiation polymerizable colipid, a chain transfer agent, and a diagnostic agent and (3) a pharmaceutically acceptable carrier, (ii) subjecting the patient to radiation in order to destabilize the liposome and release the diagnostic agent encapsulated in or associated with the liposome, and (iii) diagnosing the disease through use of molecular imaging techniques.

In one embodiment, the radiation dosage ranges from about 50 to about 2000 rads. In another embodiment, the radiation dosage ranges from about 100 to about 3000 rads. In another embodiment the radiation dosage ranges from about 500 to about 2500 rads.

Examples of diagnostic agents include, but are not limited to, contrast agents, iodinated agents, radiopharmaceuticals, fluorescent compounds and fluorescent compounds coencapsulated with a quencher, agents containing MRS/MRI sensitive nuclides, and genetic material encoding contrast agents. Examples of molecular imaging techniques include, but are not limited to, Nuclear Magnetic Resonance (NMR), Magnetic Resonance Spectroscopy/Magnetic Resonance Imaging (MRS/MRI), X-ray/computed axial tomography (CT), Positron Emission Tomography (PET), Single-photon Emission Computed Tomography (SPECT), ultrasound, and optical based imaging techniques. Examples of conditions that can be diagnosed via liposome-encapsulated or associated diagnostic agent(s) include, but are not limited to, cancer, immune disorders, developmental disorders, and genetic disorders.

Yet, another embodiment of the present invention provides for a method of producing a liposome comprising polymerizable colipid(s). The method encompasses drying the lipids that comprise the liposome(s), hydrating the lipids with a buffer comprising agents to be encapsulated or associated in a desired molar ratio to create multilamellar vesicles. Preferably, the lipids are dried in an oxygen free environment, such as an argon stream, and the vesicles are converted into liposomes by ultrasonification or freeze-thawing-extrusion. The liposomes may be purified with gel permeation chromatography or other methods.

In one embodiment, the method comprises mixing a stable liposome-forming lipid, a radiation polymerizable colipid and a chain transfer agent, hydrating the lipids with a buffer comprising agents to be encapsulated or associated in a desired molar ratio to create multilamellar vesicles or extrude or sonicate to form liposome(s), and purifying the liposome(s).

In another embodiment, the method comprises mixing a stable liposome-forming lipid, a radiation polymerizable colipid and hydrating the lipids with a buffer comprising agents to be encapsulated or associated in a desired molar ratio to create hydrated bilayers, converting the bilayers into liposome(s), purifying the liposome(s) and introducing the oxidizing agent into the liposome.

In one embodiment, cancers are treated using the liposomes of the invention. In another embodiment, cancers that are treated with radiation can also be treated with the liposomes of the invention. In addition to its therapeutic effect, the radiation acts as a releasing agent for site specific delivery of additional therapeutic or diagnostic agents by the liposome. In other embodiment, solid tumors can be treated and/or diagnosed using the liposomes of the invention. In yet another embodiment, systemic disease such as leukemia can be treated using the liposomes of the invention.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention is best understood when read in conjunction with the accompanying figures that serve to illustrate the preferred embodiments. It is understood, however, that the invention is not limited to the specific embodiments disclosed in the figures.

FIG. 1 provides a schematic representation of polymerizable lipids well known in the art. The circle in each icon represents the hydrophilic part of the molecule. The hydrophobic region is represented by the line(s). The polymerizable group X may be at the chain terminus in the middle of the bilayer (type A), near the middle of the hydrophobic chains (type B), attached to the hydrophilic head group (type C), or electrostatically associated with a charged lipid (type D).

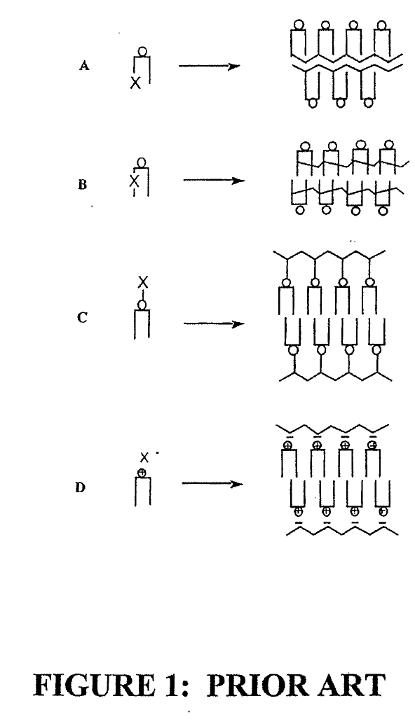

FIG. 2 depicts the structures of mono-substituted polymerizable phosphatidylcholines with various reactive groups to illustrate the instant invention. The lipid chain length and/or head group can be varied.

FIG. 3 depicts the structures of bis-substituted polymerizable phosphatidylcholines with various reactive groups to illustrate the instant invention. The lipid chain length and/or head group can be varied.

FIG. 4 shows the structures of additional polymerizable lipids, including examples of heterobifunctional lipids such as dienoyl dienyl, dienoyl sorbyl, and dienoyl acryloyl.

FIG. 5 shows examples of polymerizable phosphatidylcholines used in the liposomes of the instant invention, such as bis-SorbPC, bis-DenPC, and bis-AcrylPC.

FIG. 6 shows the synthesis for the product (10E,12E)-4,7-dimethylenetradecca-10,12-diene-1-thiol in scheme 4.

FIG. 7 shows the synthesis for the product (4E,6E,11E,13E)-pentadeca-4,6,11,13-tetraene-1-thiol in scheme 3.

FIG. 8 shows the synthesis of hydrophilic (1-ethoxy-2-ethoxy-2-ethoxy-2-methoxy-methyl)-1-hydroxy-pyridine-2-thione.

FIG. 9 shows the synthesis of the Barton ester 2-methyl-2-phenylpropionoyl-4-(1-ethoxy-2-ethoxy-2-ethoxy-2-methoxy-methyl)-1-hydroxy-pyridine-2-thione in scheme 5.

FIG. 10 shows schemes 1 and 2 for chain transfer agents based on thiols or disulfides.

FIG. 11 shows schemes 3 and 4 for unsaturated chain transfer agents such as straight chain or the branched molecule can undergo an intermolecular ring closing reaction.

FIG. 12 shows schemes 5 and 6 wherein the chain transfer agent is an ester which reacts at the sulfur to produce a carboxyl radical.

FIG. 13 shows the rate of polymerization of the liposomes in the presence of buffer.

FIG. 14 shows the rate of polymerization of the liposomes in the presence of glutathione.

FIG. 15 shows one synthesis method of the cholesteryl carboxylic acid.

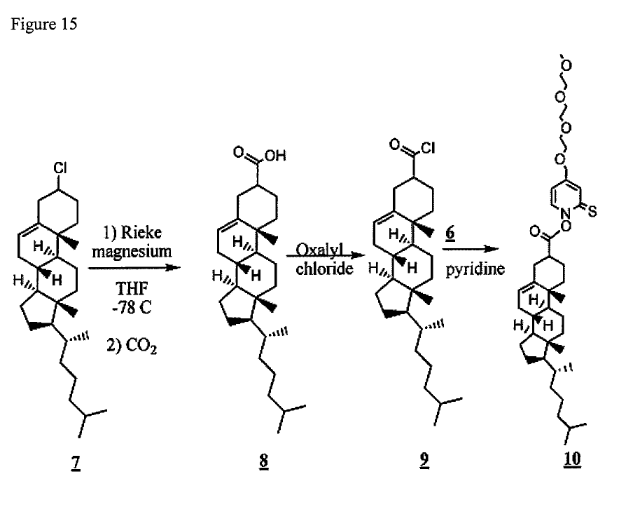

FIG. 16 shows a synthetic method for transfer agents of the present invention.

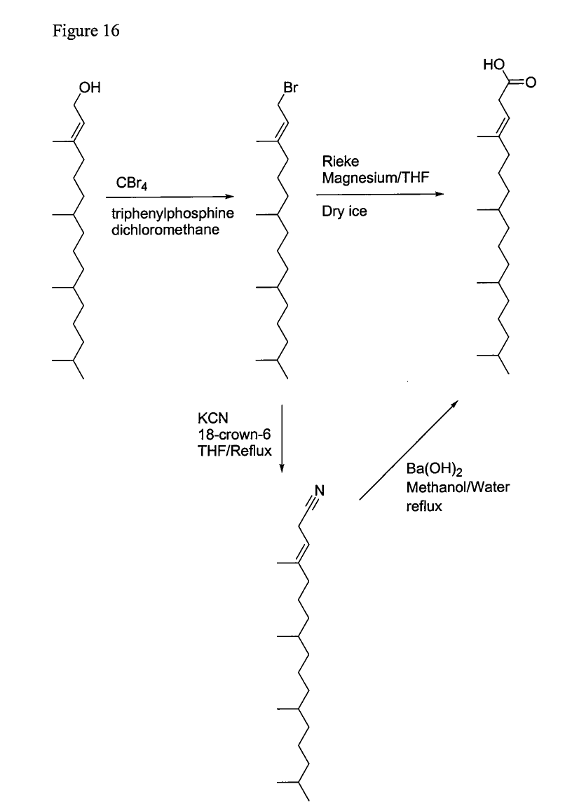

FIG. 17 shows examples of the thiol cholesterol of the present invention.

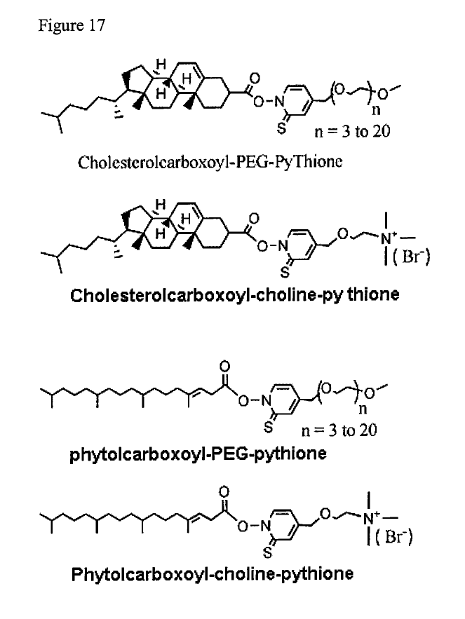

### DETAILED DESCRIPTION OF THE INVENTION

### a) Definitions and General Parameters

Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described. For purposes of the present invention, the following terms are defined below.

All publications, patents, and other reference materials referred to herein are incorporated herein by reference.

The term “liposome” refers to a microscopic vesicle comprising lipid bilayer(s). Structurally, liposomes range in size and shape from long tubes to spheres and are normally 100±10 nm in diameter, but can be as small as 25 nm and as large as 500 nm in diameter. Liposomes may contain one or more bilayer(s). Agents or molecules can be incorporated into the liposome. For example, molecules may be encapsulated in or associated with the liposome. A liposome with such encapsulated or associated agents (e.g., therapeutic or diagnostic agents) may be targeted to specific site(s) (e.g., tissue of interest such as a tumor tissue) and its contents released when appropriate. Liposomes may also be targeted to specific site(s) in vitro or in vivo through attached targeting sequences such as peptide sequences or the like.

The term “lipid”, as referred to herein, means a long-chain molecule comprised of fatty acids that may form liposomes under suitable liposome forming conditions. Examples of such lipids include, but are not limited to, phosphatidylcholine (PC), phosphatidylethanolamine (PE), phosphatidic acid (PA), phosphatidylglycerol (PG), sterol such as cholesterol, and nonnatural lipid(s) and cationic lipid(s) such as DOTMA (N-(1-(2,3-dioxyloxy)propyl)-N,N,N-trimethyl ammonium chloride).

A “polymerizable colipid”, as used herein, is any lipid that has at least one polymerizable moiety incorporated into its lipid chain(s) anywhere in the chain(s). If more than one polymerizable moiety is incorporated into the lipid chain(s), then the incorporated polymerizable moieties may be of the same or different type. For example, a polymerizable colipid may include two polymerizable moieties, such as a sorbyl and a dienoyl group, in the same colipid. Examples of polymerizable colipids include, but are not limited to, bis-SorbPC, mono-SorbPC, bis-DenPC, mono-DenPC, bis-AcrylPC, mono-AcrylPC, bis-MethacrylPC, mono-MethacrylPC, bis-VinylesterPC, mono-VinylesterPC, HeterobifunctionalPC, DiacetylenePC, MuconatePC; bis-SorbPE, mono-SorbPE, bis-DenPE, mono-DenPE, bis-AcrylPE, mono-AcrylPE, bis-MethacrylPE, mono-MethacrylPE, bis-VinylesterPE, mono-VinylesterPE, HeterobifunctionalPE, DiacetylenePE, MuconatePE; bis-SorbPG, mono-SorbPG, bis-DenPG, mono-DenPG, bis-AcrylPG, mono-AcrylPG, bis-MethacrylPG, mono-Methacryi PG, bis-VinylesterPG, mono-VinylesterPG, HeterobifunctionalPG, DiacetylenePG, MuconatePG; bis-SorbPA, mono-SorbPA, bis-DenPA, mono-DenPA, bis-AcrylPA, mono-AcrylPA, bis-MethacrylPA, mono-MethacrylPA, bis-VinylesterPA, mono-VinylesterPA, HeterobifunctionalPA, DiacetylenePA, and MuconatePA. Examples of heterobifunctional lipids are dienoyldienyl, dienoylsorbyl, and dienoylacryloyl. Examples of HeterobifunctionalPC are dienoylsorbyIPC (DenSorbPC) and dienoylacrylPC (DenAcrylPC).

The terms “main phase transition temperature”, “main transition temperature”, “transition temperature” and “Tₘ” as used interchangeably herein refers to a temperature characteristic of the lipid or polymerizable colipid. A global transition temperature refers to a transition temperature characteristic of the mixture of lipids and colipids in the liposome. If a lipid bilayer cools below a characteristic transition temperature, the lipid bilayer undergoes a phase change in which it becomes a gel-like solid state and loses its fluidity. Therefore, hydrated bilayers of a pure lipid exist in a solid-like state when the ambient/surrounding temperature is below the main phase transition temperature of the lipid. Mixtures of lipids and polymerizable colipids in liposomes are also in a gel-like solid state when the ambient temperature is near or below the Tₘ of the lowest melting lipid or colipid in the mixture. Accordingly, a liposome comprising lipids and polymerizable colipids can also exist in a gel-like solid state only when the liposome is cooled below its global main transition temperature. The lipids and polymerizable colipids are randomly distributed throughout the liposomal membrane when the ambient/environmental temperature is above the global main transition temperature of the liposome or above the transition temperatures of the lipids and the polymerizable colipids.

For liposomes administered to mammals the transition temperature of at least one lipid in the liposome is above the body temperature of the mammal. In one embodiment, the transition temperature of at least one of the lipids in the liposome is above 50° C. In another embodiment the transition temperature is above 45° C. In another embodiment, the transition temperature is above 40° C. In another embodiment, the transition temperature is above 37° C.

The terms “discrete domains of polymerizable colipids” and “discrete domains” as used herein refers to polymerizable colipids clustered together into groups of various sizes in mono- and bi-layers. Generally, formation of discrete domains is one of the methods that can be used to achieve a non-random distribution of lipids. Therefore, discrete domains may be formed by when the environmental temperature is below the Tₘ of the individual lipids. Alternatively, a liposome with discrete domains can be prepared from lipids that have a Tₘ above the patient's body temperature or if the global transition temperature of the liposome is above the patients body temperature. These discrete domains may introduce latent instability sites into the liposome. Since lipids can form immiscible mixtures of reactive and nonreactive lipids, the “discrete domains of polymerizable colipids” may exist as groups of only reactive lipids or mixtures of reactive and nonreactive lipids. Various radiation sources can polymerize the “discrete domains of polymerizable colipids”, causing shrinkage of the domains and leakage of encapsulated or associated agents from the liposome.

The term “radiation” means UV, gamma radiation and ionizing radiation. Radiation also means V, gamma radiation, and ionizing radiation delivered as an agent to the target site.

The term “therapeutic agents” means agents useful in treating or preventing an illness or disability in a patient. Such agents include, but are not limited to, chemotherapeutics, biological response modifiers, biological cofactors, pharmaceuticals and radio-pharmaceutical, cell toxins, radiation sensitizers and genetic materials. Therapeutic agents may include small organic molecules, peptides, antibodies, and nucleic acids.

The term “diagnostic agents” means agents which act to identify a disease or illness or condition in a patient. Examples of diagnostic agents include, but are not limited to, contrast agents, iodinated agents, radio-pharmaceuticals, fluorescent compounds and fluorescent compounds co-encapsulated with a quencher, agents containing MRS/MRI sensitive nuclides.

The term “biocompatible” or “biologically acceptable” or “therapeutically acceptable” means that the compound is non-toxic to the mammal or patient and can be administered to the mammal or patient.

The term “genetic materials”, as referred to herein, encompass chromosome(s), DNA, cDNA, genomic DNA, mRNA, polynucleotide(s), oligonucleotide(s), nucleic acid(s), and any synthetic DNA and RNA sequences, comprising the natural nucleotide bases adenine, guanine, cytosine, thymine, and uracil. The term also encompasses sequences having one or more modified nucleotides. The term further includes sense and antisense nucleic acids. No limitations as to length or to synthetic origin are suggested by the use of either of these terms herein.

The term “peptide” as used herein refers to one amino acid or a polymerized amino acid such as a polypeptide comprising at least two amino acids linked by peptide bonds. The term further encompasses a peptide fragment, epitope, protein fragment, antigen, antibody, antibody fragment, or any amino acid sequence. A protein is a polypeptide which is encoded by a gene.

The term “chain transfer agent” as used herein refers to a compound, molecule or species used in polymerization, which has the ability to stop the growth of a polymer chain by yielding an atom to the active radical at the end of the growing chain. It in turn is left as a radical which can initiate the growth of a new chain.

The terms “oxidation” and “oxidizing” as used herein refers to loss of electrons from an atom, compound or molecule. The terms “oxidizing agent”, “oxidant” and “oxidizer” as used herein refers to a pharmaceutically acceptable compound, molecule or species used to cause oxidation.

The terms “disease”, “disorder” and “condition” are used interchangeably herein, and refer to any disruption of normal body function, or the appearance of any type of pathology. The etiological agent causing the disruption of normal physiology may or may not be known. Furthermore, although two patients may be diagnosed with the same disorder, the particular symptoms displayed by those individuals may or may not be identical.

As used herein, the term “cancer” refers to a disease involving cells that have the potential to metastasize to distal sites and exhibit phenotypic traits that differ from those of non-cancer cells. Cancer cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless replicative potential, and sustained angiogenesis. The term “cancer cell” is meant to encompass both pre-malignant and malignant cancer cells. Forms of cancer include carcinomas, sarcomas, adenocarcinomas, lymphomas, leukemias, etc., including solid and lymphoid cancers, head and neck cancer, e.g., oral cavity, pharyngeal and tongue cancer, kidney, breast, lung, kidney, bladder, colon, ovarian, prostate, pancreas, stomach, brain, head and neck, skin, uterine, testicular, esophagus, and liver cancer, including hepatocarcinoma, lymphoma, including non-Hodgkin's lymphomas (e.g., Burkitt's, Small Cell, and Large Cell lymphomas) and Hodgkin's lymphoma, leukemia, and multiple myeloma.

The term “treatment” refers to both therapeutic treatment and prophylactic or preventative measures, wherein the object is to prevent or slow down (lessen) the targeted pathologic condition or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already with the condition or disorder as well as those prone to have the condition or disorder or those in which the condition or disorder is to be prevented.

The term “mammal” for purposes of treatment refers to any animal classified as a mammal, including humans, domestic and farm animals, and zoo, sports, or pet animals, such as dogs, cats, cattle, horses, sheep, pigs, goats, mice, primates, rabbits, etc. Preferably, the mammal is human.

The terms “subject” or “patient,” as used herein, are used interchangeably, and can refer to any to animal, and preferably a mammal, that is the subject of an examination, treatment, analysis, test or diagnosis. In one embodiment, humans are a preferred subject. A subject or patient may or may not have a disease or other pathological condition. The term “effective amount” is an amount sufficient to effect beneficial or desired therapeutic (including preventative) results. An effective amount can be administered in one or more administrations.

The term “carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®

### b) Design of the Liposomes

The instant invention describes the use of radiation to polymerize lipids in a manner that destabilizes the liposomes and causes the release of their encapsulated or associated contents at specific target sites. The radiation generates free radicals which act on the polymerizable colipids to polymerize the liposome, thereby destabilizing the liposome and releasing the releasable agent. In summary, the present invention provides a novel system and method for the release of liposome-encapsulated or associated diagnostic or therapeutic agents. Hence, radiation sensitive liposomes are particularly suitable for diagnosing and treating conditions and diseases that are responsive to liposome-encapsulated or associated agents, such as cancer, immune disorders, developmental disorders, genetic disorders, and the like.

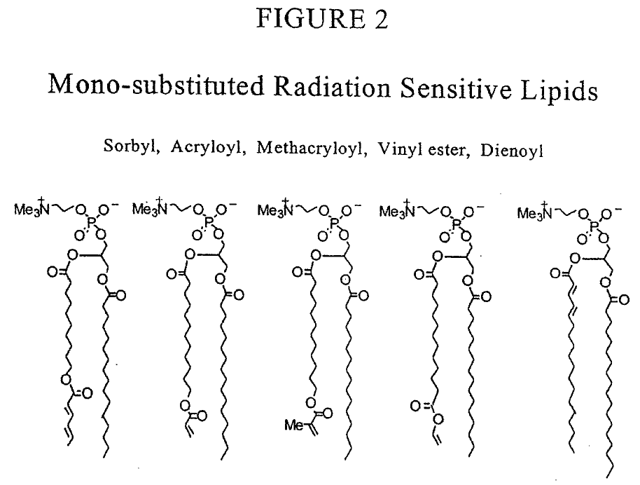

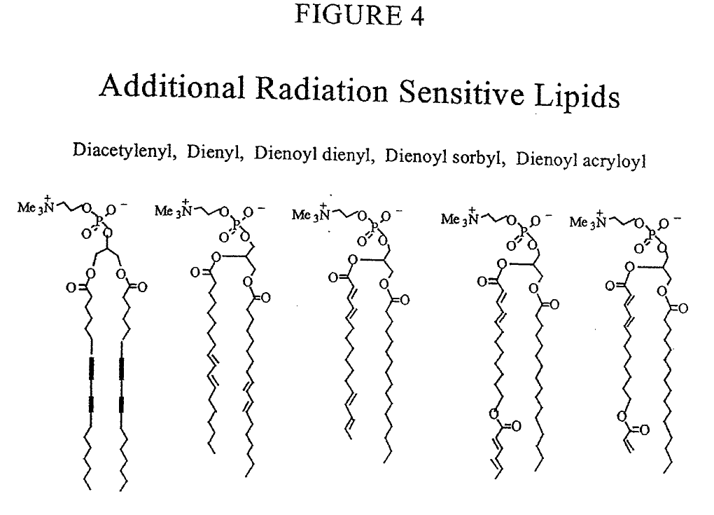

The liposomes are composed of lipids and polymerizable colipids. Lipids are long-chain molecules comprised of fatty acids that may form liposomes under suitable liposome forming conditions. Examples of lipids include, but are not limited to, phosphatidylcholine (PC), phosphatidylethanolamine (PE), phosphatidic acid (PA), phosphatidylglycerol (PG), sterol such as cholesterol, and nonnatural lipid(s) and cationic lipid(s) such as DOTMA. A polymerizable colipid is any lipid that has at least one polymerizable moiety incorporated into its lipid chain(s) anywhere in the chain(s). If more than one polymerizable moiety is incorporated into the lipid chain(s), then the incorporated polymerizable moieties may be of the same or different types. Examples of polymerizable colipids include, but are not limited to, mono-lipids with polymerizable moieties, bis-lipids with polymerizable moieties, and mixtures of mono- and bis-lipids with polymerizable moieties. More specifically, polymerizable colipid(s) include, but are not limited to, mono-, bis-, and heterobifunctional, diacetylenyl, acryloyl, methacryloyl, dienoyl, dienyl, sorbyl, muconyl, styryl, vinyl, and lipoyl colipid(s) (see FIGS. 2, 3, and 4). Heterobifunctional colipids include, but are not limited to, dienoyl dienyl, dienoyl sorbyl, and dienoyl acryloyl colipids (see FIG. 4).

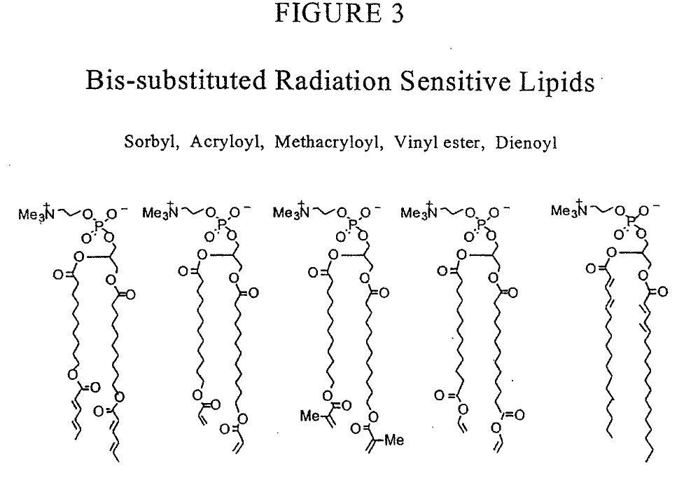

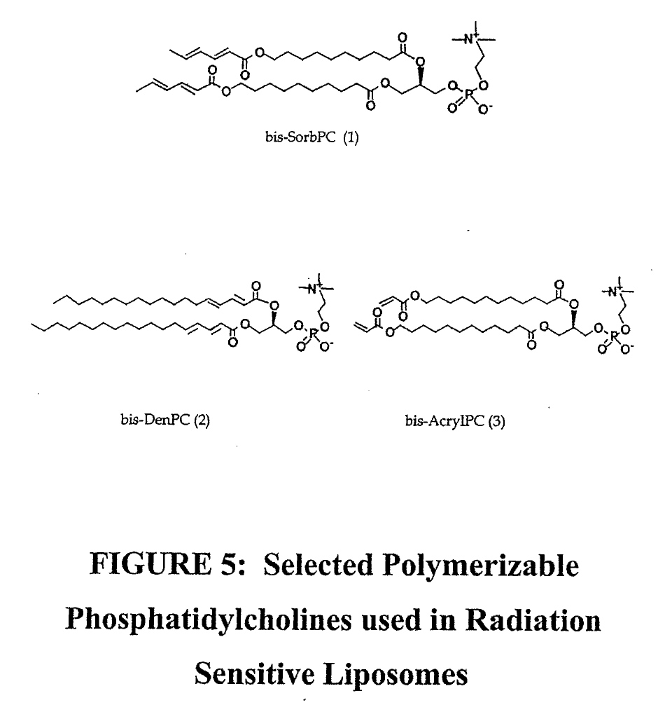

Polymerizable moieties may be incorporated into the lipid chains (see FIGS. 4 and 5). For example, bis-SorbPC and mono-SorbPC have at least one polymerizable sorbyl moiety associated with the end of both or only one of their lipid chains, respectively (see FIGS. 2 and 3). Bis-DenPC contains at least one polymerizable dienoyl moiety, while bis-AcrylPC contains at least one reactive acryloyl moiety (see FIG. 5). Alternatively, a polymerizable colipid may include two different polymerizable moieties, such as sorbyl and dienoyl group, in the same colipid.

Polymerizable colipids include, but are not limited to, bis-SorbPC, mono-SorbPC, bis-DenPC, mono-DenPC, bis-AcrylPC, mono-AcrylPC, bis-MethacrylPC, mono-MethacrylPC, bis-VinylesterPC, mono-VinylesterPC, HeterobifunctionalPC, DiacetylenePC, MuconatePC; bis-SorbPE, mono-SorbPE, bis-DenPE, mono-DenPE, bis-AcrylPE, mono-AcrylPE, bis-MethacrylPE, mono-MethacrylPE, bis-VinylesterPE, mono-VinylesterPE, HeterobifunctionalPE, DiacetylenePE, MuconatePE; bis-SorbPG, mono-SorbPG, bis-DenPG, mono-DenPG, bis-AcrylPG, mono-AcrylPG, bis-MethacrylPG, mono-MethacrylPG, bis-VinylesterPG, mono-VinylesterPG, HeterobifunctionalPG, DiacetylenePG, MuconatePG; bis-SorbPA, mono-SorbPA, bis-DenPA, mono-DenPA, bis-AcrylPA, mono-AcrylPA, bis-MethacrylPA, mono-MethacrylPA, bis-VinylesterPA, mono-VinylesterPA, HeterobifunctionalPA, DiacetylenePA, MuconatePA, DAPC, and DBPC. Examples of HeterobifunctionalPC are dienoylsorbyIPC (DenSorbPC) and dienoylacrylPC (DenAcrylPC).

The composition of the liposomes may vary, for example, the liposomes may include from about 5% to about 40% polymerizable colipids, from 10% to about 50% non-bis-SorbPC polymerizable colipids, or from 20% to about 50% of non-bis-SorbPC polymerizable colipids.

Radiation polymerizes at least a fraction of the polymerizable colipids in the liposomal membrane causing leakage of encapsulated or associated agents from the liposome. For example, after about 5% of polymerizable lipids are polymerized, leakage of liposomal contents may occur. Alternatively, after about 10% of polymerizable lipids are polymerized, leakage of liposomal contents may occur. However, no limitation with respect to the amount of polymerization and polymerization rate is suggested. The amount of leakage of encapsulated or associated agents in any given liposome depends on the nature of the liposome (e.g., the liposome composition, the location of the reactive group, the nature of the reactive group, etc.) as well as the type and strength of polymerization employed (e.g., ionizing radiation).

The liposomes of the present invention may comprise chain transfer agents. In one aspect of the invention, the chain transfer agent can be an amphiphilic chain transfer agents which will react with hydroxyl radicals to produce a stable hydrophobic radical that can initiate sorbyl polymerization in the hydrophobic interior of the lipid bilayer. In another aspect of the invention, the chain transfer agent includes, but not limited to, a cleavable hydrophilic group wherein the hydrophilic group is cleaved upon exposure to the radiation. In yet another aspect of the invention, the chain transfer agent is located near the liposome surface. In yet another aspect of the invention, the chain transfer agent can also be located up to about 4° A from the liposome surface, or up to about 3° A from the liposome surface.

In one aspect of the invention, the chain transfer agent is a thiol chain transfer agent. In another aspect of the invention, the thiol chain transfer agent is an unsaturated thiol. In yet another aspect of the invention, the thiol chain transfer agent is a branched thiol. Examples of thiol chain transfer agents include but are not limited to: DDM (dodecyl mercaptan), (4E,6E,11E,13E)-pentadeca-4,6,11,13-tetraene-1-thiol, (10E,12E)-4,7-dimethylenetetradeca-10,12-diene-1-thiol.

In another aspect of the invention, the chain transfer agent is an amphiphilic non-thiol reagent. Examples of amphiphilic non-thiol chain transfer agents include but are not limited to: halocarbons such as carbon tetrachloride, tertiary amines such as N,N-dimethyldodecyl amine and 2-hexadecanone.

In one aspect of the invention, the chain transfer agent is selected from the group consisting of: CH₃(CH₂)ₙSH, wherein n is 7 to 13; and HSCH₂CH═C(CH₃)[(CH₂)₃CH(CH₃)]ₚCH₃, wherein p is 1 to 3. In another aspect of the invention, the chain transfer agent is a Barton ester, which includes but not limited to:

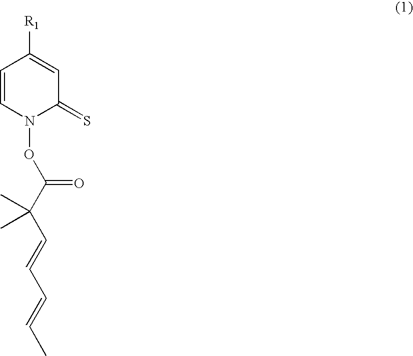

wherein R₁ is a hydrophilic group selected from —(CH₂)ₚCOOH, p is 1 to 4; —(CH₂)ₚN+(CH₃)₃, p is 1 to 4; or —(CH₂CH₂O)_{w}COOH, w is 3 to 20.

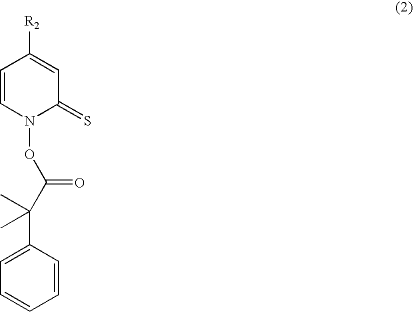

wherein R₂ is a hydrophilic group selected from —CH₂)ₚCOOH, p is 1 to 4; —(CH₂)ₚN+(CH₃)₃, p is 1 to 4; or —CH₂CH₂O)_{w}COOH, w is 3 to 20.

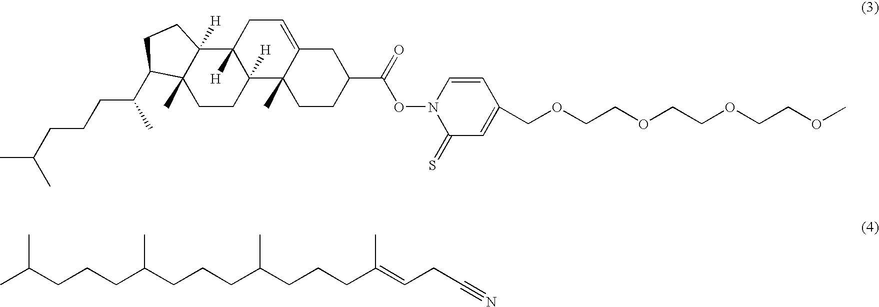

In one aspect of the invention, the chain transfer agent is a thiol cholesterol. Examples of thiol cholesterol include but are not limited to:

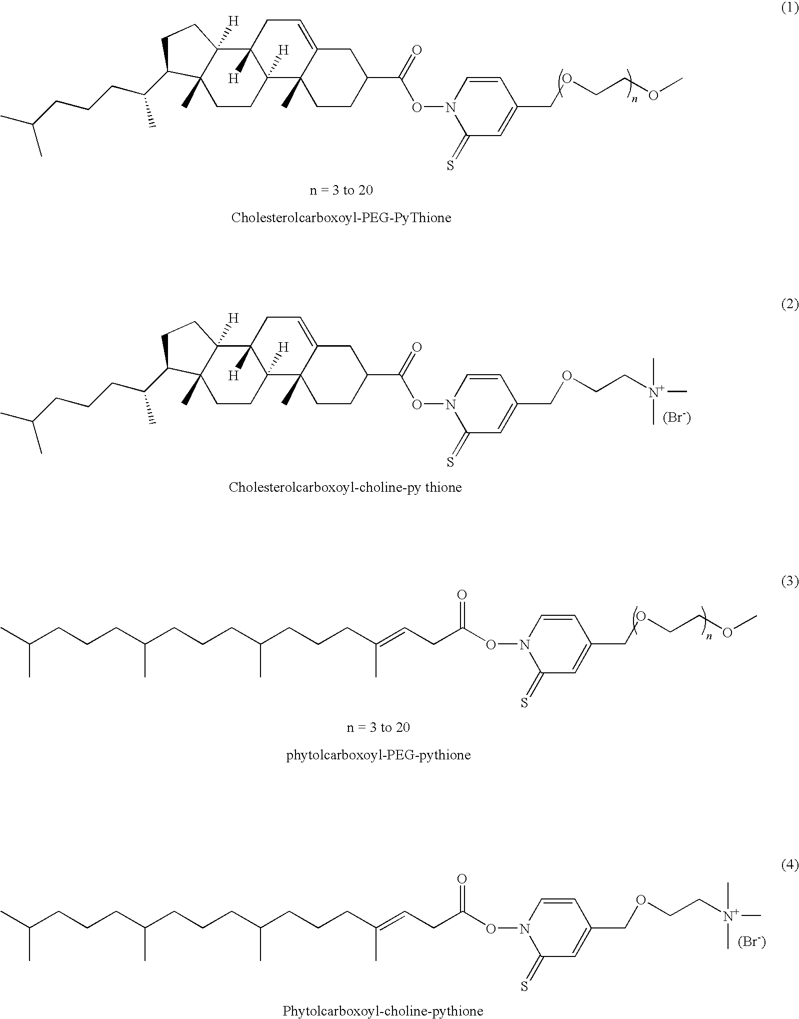

Methods of making the thiol cholesterol chain transfer agent include the following:

### 4-(bromomethyl)-pyridine-1-oxide (2)

4-methyl-pyridine-1-oxide, (1 eq), N-bromo-succinimide (1.2 eq.), and AIBN are combined in benzene. A reflux condenser is fitted to the flask, and the system is purged with argon. The solution is brought to reflux with magnetic stirring and allowed to reflux for 8 hr. Succinimide is removed by vacuum filtration and the solvent is evaporated under reduced pressure. The product is purified by crystallization from the appropriate solvent or by chromatography in a slightly acidic medium. (March, 1992 #479)

### 4-(1-ethoxy-2-ethoxy-2-ethoxy-2-methoxy-methyl)-pyridine-1-oxide (3)

mono-methoxy triethyleneglycol (1.2 eq) is dissolved in dry THF. Potassium hydride (1.4 eq) is added, and the solution is stirred. The solution will turn purple. 4-(bromomethyl)-pyridine-1-oxide (2) (1 eq.) is added, and the reaction is stirred at room temperature under argon for 3 hours. Water (1 eq) and HCl (0.5 eq) are added. The solvent is removed under reduced pressure, and the product is purified by chromatography on silica gel with ethyl acetate as a solvent. (March, 1992 #479) Other chain lengths of mono-methoxy poly(ethylene)glycol can be used at this step instead the trimer to obtain different product molecular weights and hydrophilicities.

### 4-(1-ethoxy-2-ethoxy-2-ethoxy-2-methoxy-methyl)-2-bromo-pyridine-1-oxide

(4)

### 4-(1-ethoxy-2-ethoxy-2-ethoxy-2-methoxy-methyl)-pyridine-1-oxide is dissolved in dry THF and cooled to −65° C. Butyl lithium (1 eq.) is added and the reaction is stirred for 10 minutes. Bromine (1 eq) is then added and the solution is allowed to warm to room temperature. The solution is filtered and the filtrate is evaporated at reduced pressure. The product is separated by chromatography on silica gel. (Abramovitch, 1972 #538)

### 4-(1-ethoxy-2-ethoxy-2-ethoxy-2-methoxy-methyl)-2-isothiourea-pyridine-1-oxide hydrobromide (5)

### 4-(1-ethoxy-2-ethoxy-2-ethoxy-2-methoxy-methyl)-2-bromo-pyridine-1-oxide (1 eq.) and thiourea (1 eq.) are dissolved in ethyl alcohol and brought to reflux for 1 hour. The product is separated by precipitation and vacuum filtration. (Shaw, 1950 #527)

### 4-(1-ethoxy-2-ethoxy-2-ethoxy-2-methoxy-methyl)-1-hydroxy-pyridine-2-thione (6)

4-(1-ethoxy-2-ethoxy-2-ethoxy-2-methoxy-methyl)-2-isothiourea-pyridine-1-oxide hydrobromide (1 eq.) is dissolved in water with sodium carbonate (1.9 eq.) and allowed to stand at 25° C. for 4 hours. The solution is then acidified. The product is collected by filtration or by extraction into chloroform. (Shaw, 1950 #527)

### (8S,9S,10R,13R,14S,17R)-2,3,4,7,8,9,10,11,12,13,14,15,16,17-tetradecahydro-10,13-dimethyl-17-((R)-6-methylheptan-2-yl)-1H-cyclopenta[a]phenanthrene-3-carboxylic acid (Cholesteryl-carboxilic acid)

Cholesteryl chloride (4 g, 10 mmole) is dissolved in dry THF (100 ml) and transferred into a pressure releasing addition funnel. Rieke magnesium (490 mg, 20 mmole) is suspended in 100 ml dry THF under argon in a 500 ml flask and cooled to −78 C in a dry ice bath. The solution of cholesteryl chloride is added dropwise, and the reaction mixture is removed from the dry ice bath and allowed to reach room temperature over 60 minutes.

Excess dry ice is added to the reaction mixture, and allowed to react until all bubbling stops. The solvent is removed under reduced pressure. The solid is dissolved in chloroform and washed with 1N HCL. Column chromatography on normal silica, followed by crystallization yields pure cholesteryl-carboxilic acid.

(Cholesteryl-Carboxoyl Chloride)

Oxlalyl chloride (1.9 g, 15 mmole) is added directly to cholesteryl carboxylic acid (4 g, 9.7 mmole), and the reaction mixture is stirred for 4 hr. Dry toluene (20 ml is added, and the solvent is removed under reduced pressure, with the use of a base trap.

### 1-Cholesteryl-carboxoyl-4-(1-ethoxy-2-ethoxy-2-ethoxy-2-methoxy-methyl-1-oxo-pyridine-2-thione

4-(1-ethoxy-2-ethoxy-2-ethoxy-2-methoxy-methyl)-1-hydroxy-pyridine-2-thione (1 eq) and pyridine (2 eq) are combined in dry dichloromethane under argon. The solution is cooled to 0° C. Cholesteryl-carboxoyl chloride is added and the solution is slowly allowed to warm to room temperature and stirred for 4 hours under argon. The reaction mixture is filtered and the solvent is evaporated from the filtrate under reduced pressure. The product is purified by solid phase extraction from a cation exchange resin followed by chromatography. (Sells, 1994 #353)

### (E)-1-bromo-3,7,11,15-tetramethylhexadec-2-ene

Phytol (3 g, 10 mmole) is combined with carbon tetrabromide (4.6 g, 14 mmole) under argon in dry dichloromethane (100 ml) The solution is cooled to 0° C. and triphenyl phosphine (7.3 g, 28 mmole) is added. The solution is allowed to reach room temperature, and stirred over night

Filtration through silica gel followed by evaporation of the solvent yields pure (E)-1-bromo-3,7,11,15-tetramethylhexadec-2-ene

### (E)-4,8,12,16-tetramethylheptadec-3-enoic acid

Method 1

(E)-1-bromo-3,7,11,15-tetramethylhexadec-2-ene (g, 10 mmole) is dissolved in dry THF (100 ml) and transferred into a pressure releasing addition funnel. Rieke magnesium (490 mg, 20 mmole) is suspended in 100 ml dry THF under argon in a 500 ml flask and cooled to −78 C in a dry ice bath. The solution of (E)-1-bromo-3,7,11,15-tetramethylhexadec-2-ene is added dropwise, and the reaction mixture is removed from the dry ice bath and allowed to reach room temperature over 60 minutes.

Excess dry ice is added to the reaction mixture, and allowed to react until all bubbling stops. The solvent is removed under reduced pressure. The solid is dissolved in chloroform and washed with 1N HCL. Column chromatography on normal silica, followed by crystallization yields pure (E)-4,8,12,16-tetramethylheptadec-3-enoic acid

Method 2

### (E)-4,8,12,16-tetramethylheptadec-3-enenitrile

(E)-1-bromo-3,7,11,15-tetramethylhexadec-2-ene (g, 10 mmole) is dissolved in dry THF (200 ml) Potassium cyanide (15 mmole) and 18-crown 6 are added. The suspension is allowed to reflux under argon for 24 hr. The solution is filtered and the solvent removed under reduced pressure. The solid residue is re-dissolved in chloroform and washed with saturated aqueous potassium bicarbonate followed by water. The solution is dried with brine followed by magnesium sulfate. Removal of the solvent under reduced pressure followed by chromatography on medium silica yields pure (E)-4,8,12,16-tetramethylheptadec-3-enenitrile

### (E)-4,8,12,16-tetramethylheptadec-3-enoic acid

(E)-4,8,12,16-tetramethylheptadec-3-enenitrile (10 mmole) is dissolved in methanol (200 ml) Water is added until the solution becomes slightly cloudy. Barium hydroxide (30 mmole) is added. The solution is refluxed with stirring for 24 hr, then cooled to −20 C.

The product is collected by vacuume filtration, then combined with 60 mmole of HCL in 200 ml water. Chloroform is immediately added and then phase separated. The aqueous solution is washed 2 additional times with chloroform, and the organic layer is dried with brine followed by sodium sulfate. The solvent is removed under vacuum, and the product is further purified by column chromatography on medium silica gel.

In a preferred embodiment, the chain transfer agent is selected from the group consisting of:
1) (4E,6E,11E,13E)-pentadeca-4,6,11,13-tetraene-1-thiol;
2) (10E,12E)-4,7-dimethylenetetradeca-10,12-diene-1-thiol;
3) CH₃(CH₂)ₙSH, wherein n is 7 to 13;
4) HSCH₂CH═C(CH₃)[(CH₂)₃CH(CH₃)]ₚCH₃, wherein p is 1 to 3;

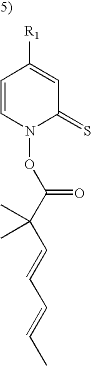

wherein R₁ is a hydrophilic group selected from —CH₂)ₚCOOH, p is 1 to 4; —(CH₂)ₚN+(CH₃)₃, p is 1 to 4; or —CH₂CH₂O)_{w}COOH, w is 3 to 20.

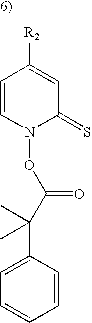

wherein R₂ is a hydrophilic group selected from —(CH₂)ₚCOOH, p is 1 to 4; —(CH₂)ₚN+(CH₃)₃, p is 1 to 4; or —(CH₂CH₂O)ₙCOOH, w is 3 to 20.

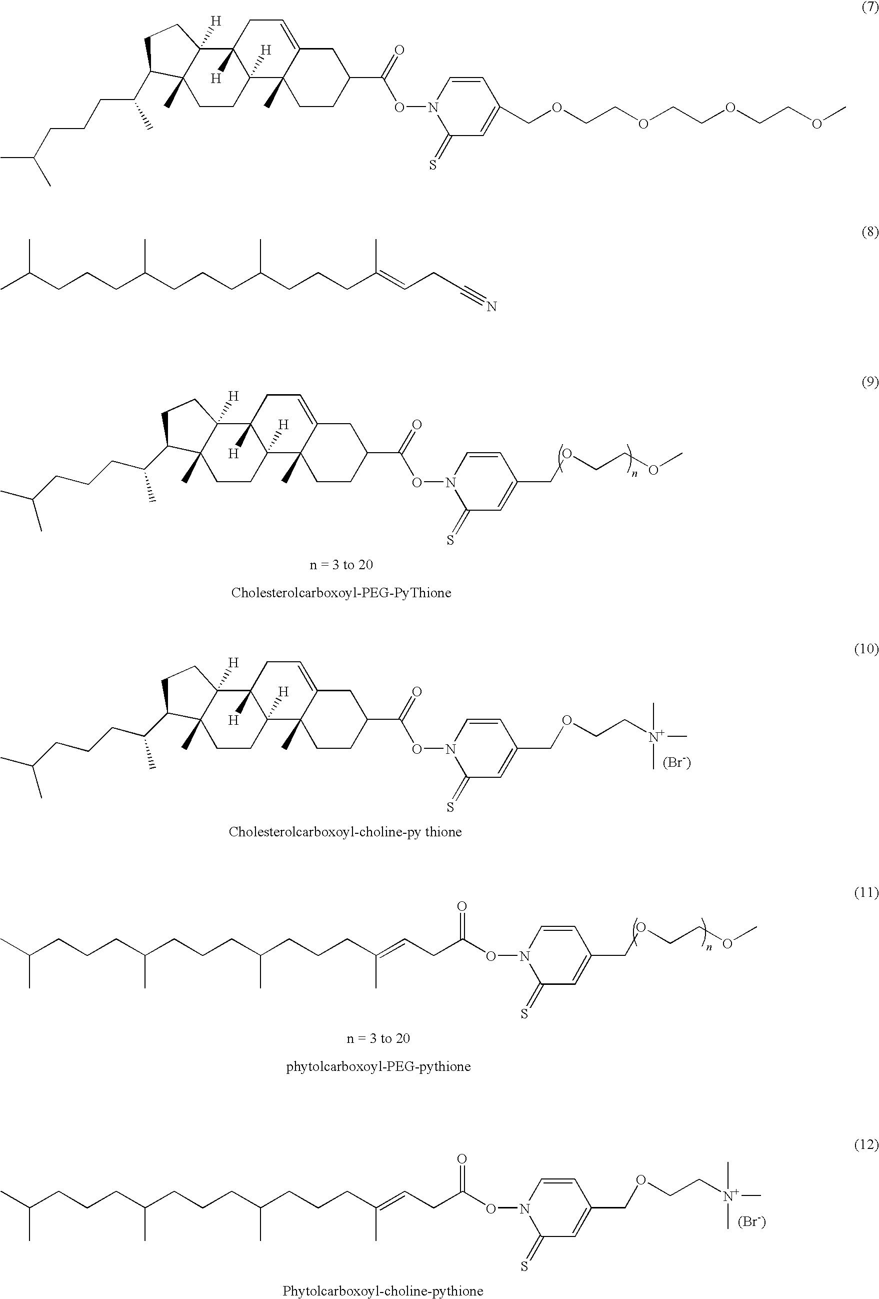

The chain transfer agent comprises about 0.01% to about 5% of total molecular weight of the liposome. In one embodiment, the chain transfer agent comprises about 0.1% to about 3% of total molecular weight of the liposome. In another embodiment, the chain transfer agent comprises about 0.1% to about 1% of total molecular weight of the liposome. In yet another embodiment, the chain transfer agent comprises about 0.5% to about 1% of total molecular weight of the liposome.

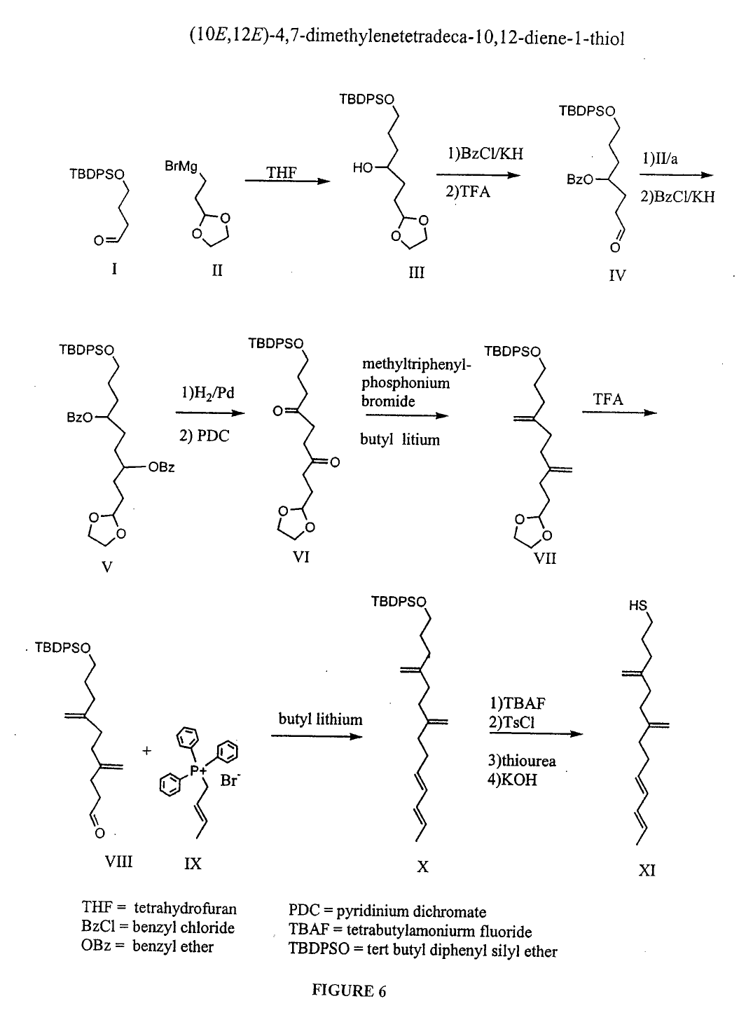

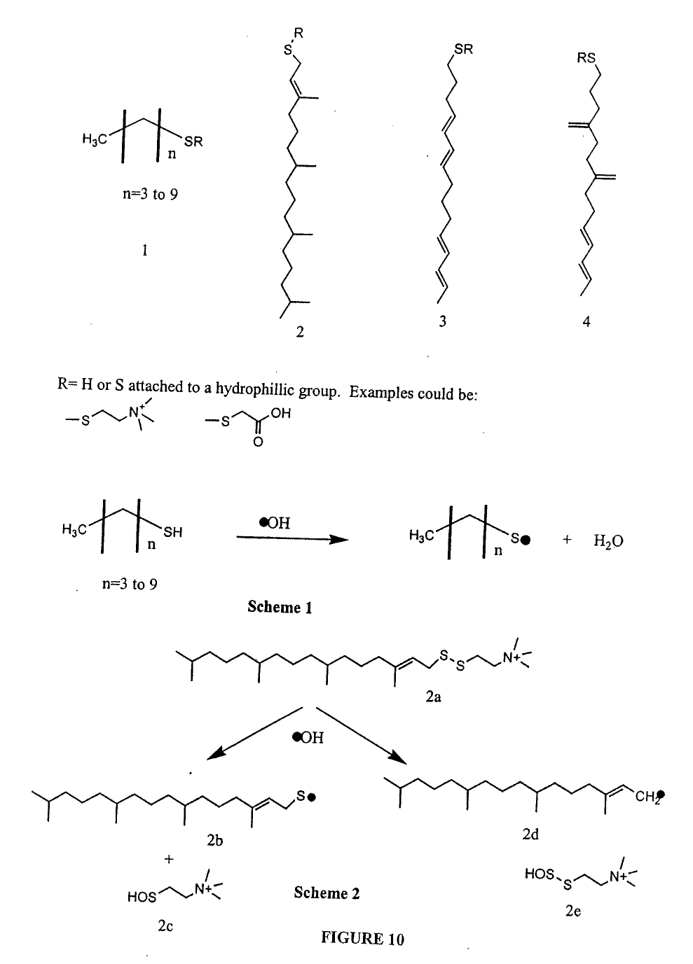

With reference to FIG. 6 and scheme 1 in FIG. 10, when exposed to hydroxyl radical generated by ionizing radiation, a thiol will react as in scheme 1 to produce an alkyl sulfide radical 1a and water.

With reference to FIG. 6 and scheme 2 in FIG. 10, an allylic disulfide such as 2a can react with the hydroxyl radical according to scheme 2 to produce either the sulfide radical 2b through sulfur-sulfur bond cleavage or the allylic radical, 2d through carbon-sulfur bond cleavage. Either type of radical may diffuse into the membrane and react with the sorbyl monomer.

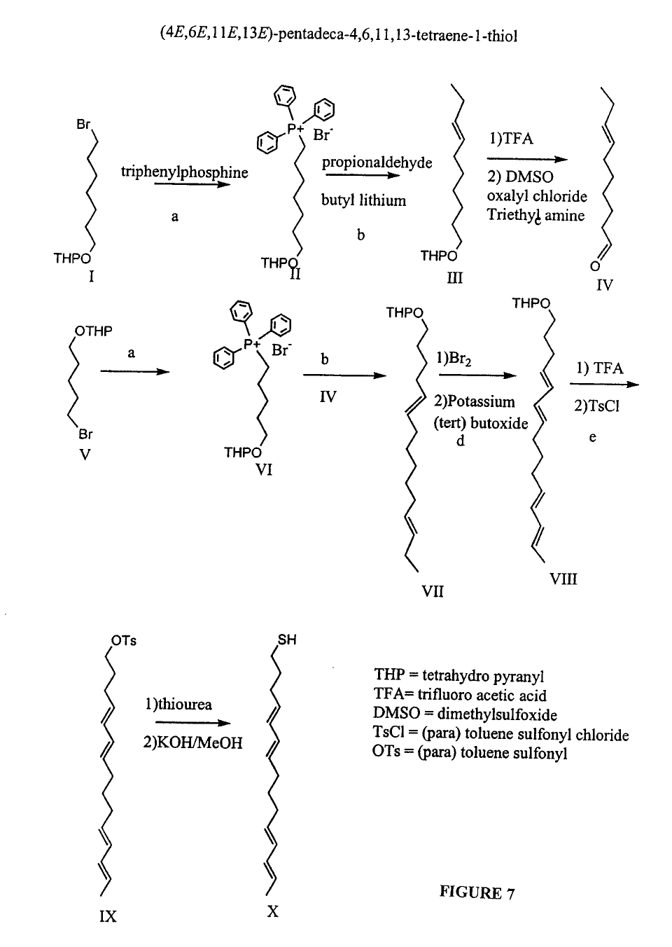

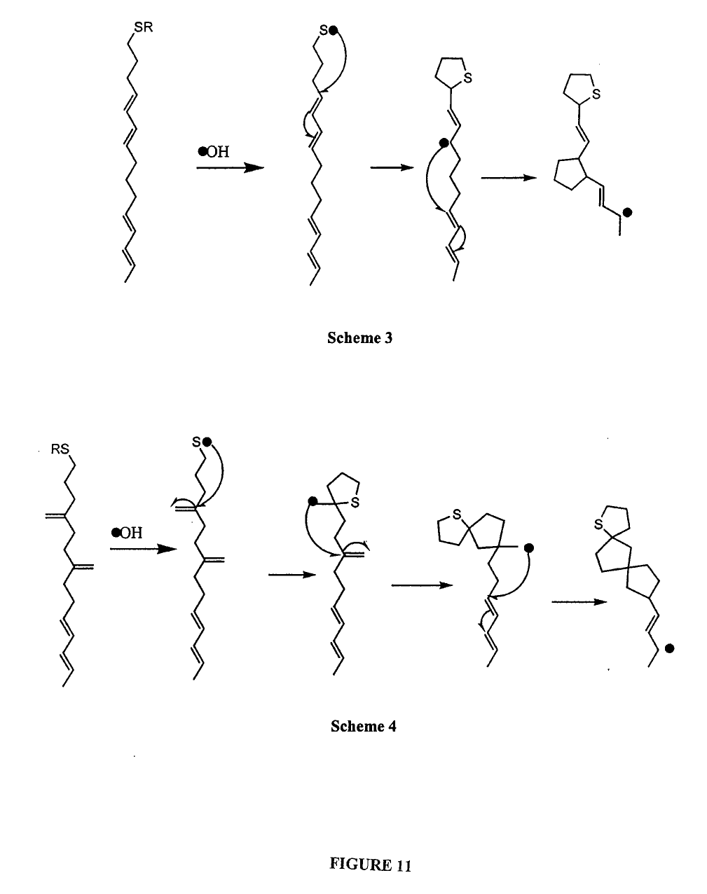

With reference to FIG. 7 and schemes 3 and 4 in FIG. 11, unsaturated chain transfer agents such as the straight chain 3 or the branched molecule 4 can undergo an intramolecular ring closing reaction which will transfer the radical into the bilayer without net diffusion of the molecule. The successive cyclizations of these molecules to form a series of 5-membered rings are shown in schemes 3 and 4.

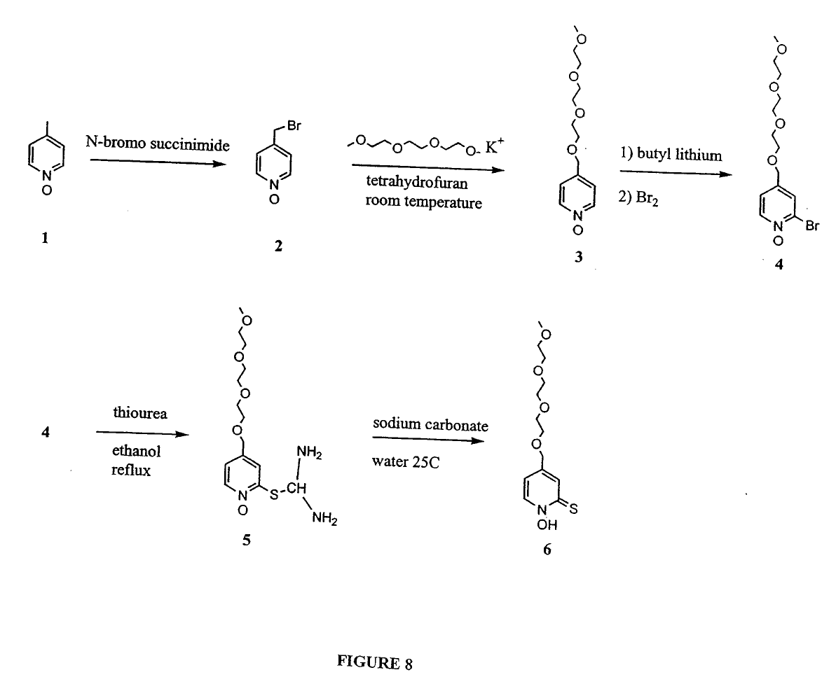

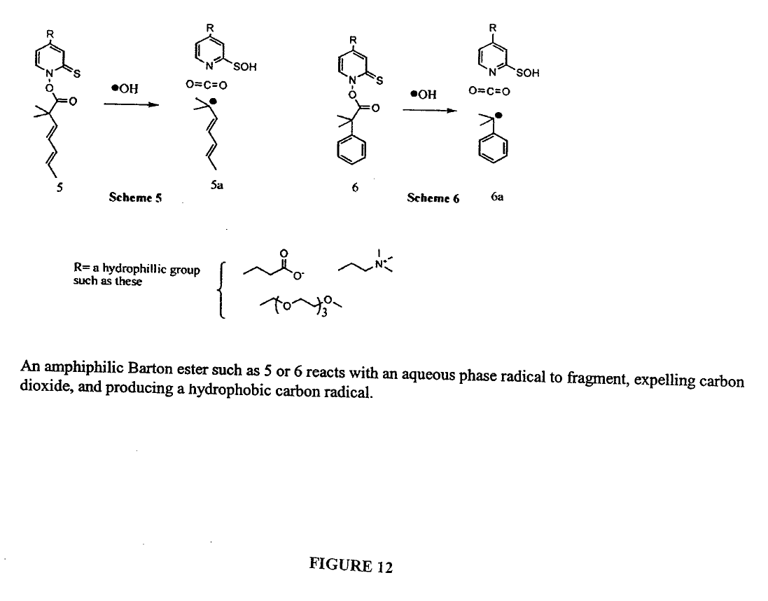

With reference to FIG. 8 and schemes 5 and 6 in FIG. 12, amphiphilic esters, such as 5 or 6, the hydroxyl radical generated by ionizing radiation reacts with the esters to produce a carboxyl radical which spontaneously decarboxylates as in schemes 5 and 6 to give resonance stabilized radicals 5a and 6a, respectively, as well as carbon dioxide and a pyridine derivative.

Certain liposomes of the present invention may comprise of oxidizing agents. In one aspect of the invention, the oxidizing agents include, but not limited to, potassium bromate, potassium persulfate (K₂S₂O₈) (or other bio-compatible persulfates), iron(III) sulfate (Fe(SO₄)₃) (or other bio-compatible Fe(III) salts), hydrogen peroxide (H₂O₂), tert-butyl hydroperoxide ((CH₃)₃COOH), and peroxy-carboxylic acids such as CH₃C(═O)OOH.

The concentration of the oxidizing agent in the solution containing the liposomes is approximately 0.05 to 0.3 M.

In one aspect of the invention, the mixture has about 5% to 60% of liposome with oxidizing agents. In yet another aspect of the invention, the mixture has about 10% to 60% of liposome with oxidizing agents. In yet another aspect of the invention, the mixture has about 20% to 60% of liposome with oxidizing agents. In yet another aspect of the invention, the mixture has about 40% to 60% of liposome with oxidizing agents. In yet another aspect of the invention, the mixture has about 50% of liposome with oxidizing agents.

The liposomes may further comprise a releasable agent. Such releasable agents may be water soluble molecules. Alternatively, liposome(s) may comprise lipid associated molecules. Hence, the liposome of the instant invention may comprise a releasable agent that is water-soluble or lipid-associated molecule. The releasable agent(s) include, but are not limited to, therapeutic agents or diagnostic agents.

Examples of therapeutic agents include, but are not limited to, chemotherapeutics, biological response modifiers, biological cofactors, pharmaceuticals and radiopharmaceuticals, cell toxins, radiation sensitizers, and genetic materials. Examples of chemotherapeutics include, but are not limited to, alkylating agents such as nitrogen mustards (e.g., chlorambucil, estramustine, mechlorethamine, melphalan); ethylenimine derivatives such as thiotepa; alkyl sulfonates such as busulfan; nitrosoureas such as carmustine (BCNU), lomustine (CCNU), methyl-CCNU, and streptozocin; triazines such as dacarbazine; metal salts such as cisplatin and carboplatin; antimetabolites; folic acid analogues such as methotrexate; pyrimidine analogues such as 5-fluorouracil and floxuridine; purine analogues such as 6-mercaptopurine, 6-thioguanine, deoxycorformycin, and fludarabine; natural products such as vinca alkaloids (e.g., vinblastine and vincristine); podophyllum derivatives such as etoposide and teniposide; antibiotics such as, bleomycin, dactinomycin, doxorubicin, mithramycin, mitomycin, mitoxantrone; hormones and hormone antagonists; androgens such as halotestin, testolactone; corticosteroids such as prednisone and dexamethasone; estrogens such as dethylstilbestrol; progestins megestrol acetate and medroxyprogesterone acetate; estrogen antagonists tamoxifen and taxol; androgen antagonists such as flutamide; LHRH agonists such as leuprolide and goserelin; substituted ureas such as hydroxyurea; methylhydrazine derivatives such as procarbazine; adrenocortical suppressants such as mitotane; steroid synthesis inhibitors such as aminoglutethemide; and substituted melamines such as altretamine. Examples of biological response modifiers include, but are not limited to, interferons such as interferon alpha and interferon gamma; interleukins such as IL-2; and tumor necrosis factor. Examples of radiopharmaceuticals include, but are not limited to, meta-iodobenzylguanidine (MIBG) and ¹¹¹In-labeled somatostatin analog [diethylenetriaminepentaacetic acid (DTPA)-DPhe1]-octreotide. Examples of cell toxins include, but are not limited to, tirapazamine and others. Examples of radiation sensitizers include, but are not limited to, nitroimidazoles such as misonidazole, etanidazole, metronidazole, and nimorazole; docetaxel, paclitaxel, idoxuridine, fludarabine, gemcitabine, and taxanes. Examples of genetic materials include, but are not limited to, chromosome(s), DNA, cDNA, genomic DNA, mRNA, polynucleotide(s), oligonucleotide(s), nucleic acid(s), any synthetic DNA and RNA sequences, and sense and antisense nucleic acid(s). The liposomes of the instant invention can deliver therapeutic agents to specific sites in the body. For example, the liposomes can selectively localize anti-cancer drugs or other agents at a tumor site, resulting in markedly reduced toxicity in addition to improved therapeutic activity due to higher drug levels being delivered to the tumor.

The ratio of therapeutic agent to lipid may be up to about 20% by weight, or from about 0.001% to about 0.1%. Examples of diagnostic agents include, but are not limited to, contrast agents, iodinated agents, radiopharmaceuticals, fluorescent compounds and fluorescent compounds coencapsulated with a quencher, agents containing MRS/MRI sensitive nuclides, and genetic material encoding contrast agents. Examples of diagnostic agents include, but are not limited to, contrast agents, iodinated agents, radiopharmaceuticals, antibodies, fluorescent compounds and fluorescent compounds coencapsulated with a quencher, agents containing MRS/MRI sensitive nuclides, and genetic material encoding contrast agents. Examples of contrast agents include, but are not limited to, gadolinium-diethylenetriaminepentaacetic acid (GdDTPA; Magnavist), aka gadopentetate dimeglumine, gadoteridol (ProHance), gadodiamide, gadoterate meglumine, gadobenate dimeglumine (Gd-BOPTA/Dimeg; MultiHance), mangafodipir trisodium (Mn-DPDP), ferumoxides, paramagnetic analogue of doxorubicin, and ruboxyl (Rb). Examples of iodinated agents include, but are not limited to, diatrizoate (3,5-di(acetamido)-2,4,6-triiodobenzoic acid), iodipamide (3,3′-adipoyl-diimino-di(2,4,6-triiodobenzoic acid), acetrizoate [3-acetylamino-2,4,6-triiodobenzoic acid], aminotrizoate [3-amino-2,4,6-triiodobenzoic acid]), and iomeprol. Examples of radiopharmaceuticals include, but are not limited to, fluorine-18 fluorodeoxyglucose ([¹⁸F]FDG), Tc-99m Depreotide, carbon-11 hydroxyephedrine (HED), [18F]setoperone, [methyl-¹¹C]thymidine, 99 mTc-hexamethyl propyleneamine oxime (HMPAO), 99 mTc-L, L-ethylcysteinate dimer (ECD), 99 mTc-sestamibi, thallium 201, 1-131 metaiodobenzylguanidine (MIBG), 123I—N-isopropyl-p-iodoaamphetamine (IMP), 99 mTc-hexakis-2-methoxyisobutylisonitrile (MIBI), 99 mTc-tetrofosmin. Examples of agents containing MRS/MRI sensitive nuclides include, but are not limited to, perfluorocarbons and fluorodeoxyglucose. Examples of genetic material encoding contrast agents include, but are not limited to, paramagnetic reporter genes such as ferredoxin; paramagnetic tag(s) on liposomal lipids such as paramagnetic chelating groups added to PEG; detectable probes; and luciferin/luciferase reporter system.

The ratio of diagnostic agent to lipid may be up to about 20% by weight, or from about 0.001% to about 0.1% by weight.

The liposomes containing the oxidizing agents may be mixed with liposomes containing other types of releasable agents, either therapeutic agents or diagnostic agents to form a mixture of liposomes. The liposomes containing a therapeutic agent or a diagnostic agent may also further comprise chain transfer agents. Liposomes comprising an oxidizing agent may not contain an easily oxidizable chain transfer agents such as thiol or tertiary amine chain transfer agents.

The liposomes are administered to the patients and the patients are exposed to radiation after a sufficient period to allow the liposomes to move to the site of radiation from the site of administration. The ionizing radiation may be gamma (γ) radiation or alpha (α) radiation or beta (β) radiation or other ionizing radiation (i.e. X-rays, brachytherapy seeds). The period of time required for the liposomes to move to the site of radiation will vary with the liposome composition and the patient.

In one embodiment the radiation is ionizing radiation. Using ionizing radiation has distinct advantages over UV or photoinitiated release. First, and most importantly, ionizing radiation is not limited by the depth of penetration as is radiant energy. Ionizing radiation can penetrate even the deepest of tissue sites. Secondly, the use of ionizing radiation as a standard treatment for various conditions and diseases relies on sources that are readily available (e.g., radiation treatment in hospitals and clinics). Furthermore, dosimetry for radiation treatment has already been carefully determined in the art which establishes immediate applicability.

In one embodiment, the liposomes comprise a steric stabilizer. The steric stabilizer may be polyethylene glycol (PEG). The attachment of PEG to lipids in the liposome increases the period of circulation of the liposome in vivo. Examples of PEG-liposome compositions are various combinations of PEG and PCs, and/or PEs, and/or PAs, and/or PGs, and/or sterols such as cholesterol, and/or normatural lipids, and/or cationic lipids.

PEG-modified PE may be incorporated into liposomes by including it with the other lipids during the formation of the liposomes. Alternatively, monomethoxy-PEG has been coupled to the outer surface of preformed liposomes, which contains some fraction of PE (Senior et al., Biochim. Biophys. Acta 1991, 1062:77-82). Regardless of the means of PEG incorporation, the inclusion of PEG ranging in size from about 1000 to 5000 daltons results in liposomes with an order of magnitude or greater increase in circulation time in the body. The useful mole fraction of PEG/PE depends on the polymer chain length. Thus 5 mole percent of PEG₁₉₀₀, wherein the 1900 indicates the number average molecular weight of the PEG, may be effective in achieving increased circulation time, whereas 15 mole percent of PEG₇₅₀ may be necessary to achieve a comparable stabilization. A minimum necessary surface coverage of the liposome is achieved at a lower mole fraction of the longer polymer.

The liposomes may include from about 1% to about 20% steric stabilizer, the liposomes comprise from 2% to about 10% of steric stabilizer or the liposomes may comprise from 2% steric stabilizer to about 5% steric stabilizer. In still another embodiment, the liposomes may include from about from about 5% to about 40% polymerizable colipids and from about 2% to about 20% steric stabilizer.

In one embodiment of the instant invention, the liposomes are comprised of PEG₂₀₀₀-dioleoylPE, cholesterol, dioleoylPC, and bis-SorbPC_{19,19}. In another embodiment of the instant invention, the liposomes are comprised of PEG₂₀₀₀-distearoylPE, cholesterol, distearoylPC, and bis-SorbPC_{19,19}. In yet another embodiment of the instant invention, the liposome(s) are comprised of PEG₂₀₀₀-distearoylPE, distearoylPC, and bis-SorbPC_{19,19}. In yet another embodiment of the instant invention, the liposome(s) are comprised of PEG-liposomes with a molar ratio of about 19% to about 30% bis-SorbPC_{19,19}, about 40% to about 63% of 1,2-Diarachidoyl-sn-Glycero-3-Phosphocholine or 1,2-Dibehenoyl-sn-Glycero-3-Phosphocholine, about 5% PEG₂₀₀₀-distearoylPE, and about 0% to about 35% cholesterol. Specific examples of PEG-liposomes include, but are not limited to, PEG-liposomes with a molar ratio of about 15% PEG₂₀₀₀-dioleoylPE, 40% cholesterol, 20% dioleoylPC, and 30% bis-SorbPC_{19,19}; PEG-liposomes with a molar ratio of about 15% PEG₂₀₀₀-distearoylPE, 40% cholesterol, 20% distearoylPC, and 30% bis-SorbPC_{19,19}; and PEG-liposomes with a molar ratio of about 5% PEG₂₀₀₀-distearoylPE, 75% distearoylPC, and 20% bis-SorbPC_{19,19}. In one preferred embodiment, a PEG-liposome having a molar ratio of about 30% distearoylPC, 30% bis-SorbPC_{19,19}, 35% cholesterol, and 5% PEG₂₀₀₀-distearoylPE is used. In another preferred embodiment, a PEG-liposome having a molar ratio of about 52% distearoylPC, 23% bis-SorbPC_{19,19}, 20% cholesterol, and 5% PEG₂₀₀₀-distearoylPE is used. In yet another preferred embodiment, a PEG-liposome having a molar ratio of about 45% distearoylPC, 20% bis-SorbPC_{19,19}, 30% cholesterol, and 5% PEG₂₀₀₀-distearoylPE is used.

Liposomal drug delivery provides advantages over viral delivery and other methods such as greater biocompatibility, less toxicity, fewer side effects, and others. Furthermore, the addition of nucleic acid material to liposomes for therapeutic purposes may trigger structural changes in the liposomes as well as the DNA. The benefits associated with such structural changes are that the DNA molecule adopts a structure that renders the molecule partially protected from extracellular and intracellular degradation. Hence, the liposomes provide a targeting as well as a protective mechanism for the encapsulated nucleic acids.

The present invention further contemplates a liposome comprising a radiation sensitive liposome and releasable agent that can be targeted to a tumor site through attachment of at least one peptide to the liposome. Peptides that target liposome(s) to tumor sites include, but are not limited to, peptide sequences, peptide fragments, antibodies, antibody fragments, and antigens. Antibodies that are specific to tumor cells can be attached to a liposome which allows for selective targeting. Such antibodies can bind to selective antigens on the tumor cells which brings the liposome into close proximity of a tumor or malignant tissue. After binding of the antibody to the tumor antigen the liposome is linked to the tumor tissue. Radiation can then trigger the release of the liposomal contents such as anti-tumor agents. Specific antigens may also be attached to the liposome. Such antigens may link attached liposomes to antibodies that are localized around tumor sites. Specific peptide sequences (e.g., epitopes) that bind to selective receptors may also be attached to the liposome, wherein any number of peptide sequences may be attached. The peptide sequence targets the liposome to a specific tissue. One or more peptides may be used to specifically target a liposome to a tumor tissue. One advantage of introducing several targeting peptides to the surface of the liposome is that, while the target affinity for a single peptide may be lower, the incorporation of multiple peptides into a liposome increases the overall likelihood of these peptides to bind to selective receptors on a tumor tissue.

### c) Production of Radiation Sensitive Liposomes

Another embodiment of the present invention provides for a method of producing a radiation sensitive liposome comprising polymerizable colipid(s) and chain transfer agents. The method encompasses drying the mixture of lipids, colipids and chain transfer agents that comprise the liposomes, hydrating the lipids with a buffer comprising agents to be encapsulated or associated in a desired molar ratio to create hydrated bilayers, converting the bilayers into liposomes, and purifying the liposomes. The encapsulated or associated agents include, but are not limited to, therapeutic and diagnostic agents.

The lipids, colipids and chain transfer agents that comprise the liposome may be dried in an oxygen-free environment such as under an argon stream, followed by drying under vacuum and weighing. The lipids in the desired molar ratio are hydrated with a buffer including the agents to be encapsulated or associated. The hydrated bilayers can then be converted into liposomes by either ultrasonication, freeze/thawing and extrusion procedures, or other conventional liposome preparation procedures. In some instances it may be preferable to load the liposomes with therapeutic agents by using a pH gradient to drive weak bases, such as doxorubicin, into the liposomes. The liposomes may be purified by elution such as a gel permeation chromatography column with an isoosmotic buffer to remove unencapsulated or unassociated agents. Other purification methods can be used as well. The liposome size distributions can be measured by dynamic light scattering. The liposomes can also be imaged by electron microscopy. Liposomes are normally 100±10 nm in diameter, but can be as small as 25 nm and as large as 500 nm in diameter.

### d) Treatment with Radiation Sensitive Liposomes

Lipid assemblies, such as liposomes, are arrays of noncovalently associated amphiphiles, i.e., supramolecular assemblies. They can be classified as supported or self-supported assemblies. The polymerization of liposomes can lock in preexisting lipid domains or create lipid domains from random mixtures, depending on the nature of the polymerizable amphiphile. Lipids can form an unpolymerized immiscible mixture of reactive and nonreactive lipids in mono- and bi-layers. In contrast, polymerization of polymerizable colipids can effectively induce the phase separation of unreactive lipids from the growing polymeric domains. Hence, lipid domains can endow liposomes with latent instability sites. Therapeutic doses of ionizing radiation can then substantially enhance the release of encapsulated water soluble or lipid associated molecules from radiation sensitive liposomes. These conditions are suitable for the destabilization of radiation sensitive liposomes that are optionally sterically stabilized.

One embodiment of the instant invention describes the use of ionizing radiation in order to polymerize colipids clustered in discrete domains in a manner that destabilizes the liposomes and thereby causes leakage of their encapsulated contents such as therapeutic or diagnostic agents, wherein the leakage between or around the discrete domain boundaries is due to the shrinkage of the domains. Upon exposure to ionizing radiation these lipids polymerize and allow leakage of liposomal contents throughout the liposome. Hence, destabilization can be achieved by polymerization of reactive colipids in the lipid bilayers of the liposomes. Polymerizable colipids have different reactivities that control the initial rate of polymerization, extent of polymerization, and inhibition by oxygen. The interaction of ionizing radiation with water produces radical species that can initiate radical chain polymerizations. The use of ionizing radiation to trigger release of liposomal contents at the target site(s) in vivo or in vitro has several distinct advantages. Most importantly, ionizing radiation is not limited by the depth of penetration or the thickness of the specimen since it can penetrate through all layers of tissue. Furthermore, the use of ionizing radiation provides an efficient and convenient means of treating cancer and other diseases, due to its ease of integration into currently available radiation based clinical methods.

Therapeutic doses of ionizing radiation can substantially enhance the release of encapsulated water soluble or lipid associated molecules from radiation sensitive liposomes. These conditions are suitable for the destabilization of radiation sensitive liposomes that are sterically stabilized. The enhanced depth of penetration of ionizing radiation is particularly suitable for therapeutic purposes. Moreover, because most medical centers have experience with focused ionizing radiation, the radiation induced destabilization of liposomes offers a spatially and temporally selective method to deliver and release agents for medical therapy and diagnostics.

Radiation sensitive liposomes possess the ability to localize at tumor sites due to increased permeability of the vasculature near tumor associated areas. The release of encapsulated or associated agents from liposomes may occur passively or may be stimulated or induced. For example, ionizing radiation can stimulate the release of liposomal contents. Furthermore, liposomal delivery to target cells may be accomplished through leakage (passive or induced) or through endocytosis. In endocytosis, liposomes contact the cell membrane, form a vesicle within an endosome, and eventually fuse with other organelles to release their contents. Therapeutic doses of ionizing radiation substantially enhance the release of encapsulated water soluble molecules or lipid associated molecules from the liposomes. The enhanced depth of penetration of ionizing radiation overcomes the limitation of previous methods which rely on UV light to destabilize liposomes since UV light does not penetrate beyond the epidermal layer of the body. Thus, these conditions are suitable for the destabilization of radiation sensitive liposomes, particularly for therapeutic and diagnostic purposes.

In one embodiment, the instant invention provides for a method of treating a condition responsive to a liposome-encapsulated or associated therapeutic agent, comprising the steps of (i) administering to a patient a pharmaceutical composition comprising a liposomal delivery system and releasable agent such as a therapeutic agent, wherein the releasable agent is encapsulated in or associated with the liposome, and a pharmaceutically acceptable carrier or diluent; and (ii) subjecting the patient to radiation in order to destabilize the liposome and release the therapeutic agent encapsulated in or associated with the liposome. In one embodiment, the radiation dosage ranges from about 50 to about 2000 rads.

In another embodiment, the radiation dosage ranges from about 100 to about 3000 rads. In another embodiment, the radiation dosage ranges from about 500 to about 2500 rads. Examples of conditions that are responsive to liposome-encapsulated or associated therapeutic agent(s) include, but are not limited to, cancer, immune disorders, developmental disorders, and genetic disorders. Preferably, the liposome-encapculated or associated therapeutic agent(s) can be used to treat solid oncogenic growth: (1) solid tumors such as adrenocortical carcinoma, carcinoma, colorectal carcinoma, desmoplastic small round cell tumor, Germ Cell tumor, sarcoma, hepatoblastoma, hepatocellular carcinoma, melanoma, neuroblastoma, non-rhabdomyosacrcoma soft tissue sarcoma, osteosarcoma, peripheral primitive neurorectodermal tumor, retinoblastoma, rhabdomyosarcoma, Wilms tumor; (2) endocrine tumors such as localized lyphomas. Examples of therapeutic agents include, but are not limited to, chemotherapeutics, biological response modifiers, biological cofactors, pharmaceuticals and radiopharmaceuticals, cell toxins, radiation sensitizers, and genetic materials. Examples of chemotherapeutics include, but are not limited to, alkylating agents such as nitrogen mustards (e.g., chlorambucil, estramustine, mechlorethamine, melphalan); ethylenimine derivatives such as thiotepa; alkyl sulfonates such as busulfan; nitrosoureas such as carmustine (BCNU), lomustine (CCNU), methyl-CCNU, and streptozocin; triazines such as dacarbazine; metal salts such as cisplatin and carboplatin; antimetabolites; folic acid analogues such as methotrexate; pyrimidine analogues such as 5-fluorouracil and floxuridine; purine analogues such as 6-mercaptopurine, 6-thioguanine, deoxycorformycin, and fludarabine; natural products such as vinca alkaloids (e.g., vinblastine and vincristine); podophyllum derivatives such as etoposide and teniposide; antibiotics such as, bleomycin, dactinomycin, doxorubicin, mithramycin, mitomycin, mitoxantrone; hormones and hormone antagonists; androgens such as halotestin, testolactone; corticosteroids such as prednisone and dexamethasone; estrogens such as dethylstilbestrol; progestins megestrol acetate and medroxyprogesterone acetate; estrogen antagonists tamoxifen and taxol; androgen antagonists such as flutamide; LHRH agonists such as leuprolide and goserelin; substituted ureas such as hydroxyurea; methylhydrazine derivatives such as procarbazine; adrenocortical suppressants such as mitotane; steroid synthesis inhibitors such as aminoglutethemide; and substituted melamines such as altretamine. Examples of biological response modifiers include, but are not limited to, interferons such as interferon alpha and interferon gamma; interleukins such as IL-2; and tumor necrosis factor. Examples of radiopharmaceuticals include, but are not limited to, meta-iodobenzylguanidine (MIBG) and ¹¹¹In-labeled somatostatin analog [diethylenetriaminepentaacetic acid (DTPA)-DPhe1]-octreotide. Examples of cell toxins include, but are not limited to, tirapazamine and others. Examples of radiation sensitizers include, but are not limited to, nitroimidazoles such as misonidazole, etanidazole, metronidazole, and nimorazole; docetaxel, paclitaxel, idoxuridine, fludarabine, gemcitabine, and taxanes. Examples of genetic materials include, but are not limited to, chromosome(s), DNA, cDNA, genomic DNA, mRNA, polynucleotide(s), oligonucleotide(s), nucleic acid(s), any synthetic DNA and RNA sequences, and sense and antisense nucleic acid(s). The liposomes of the instant invention can deliver therapeutic agents to specific sites in the body. For example, the liposomes can selectively localize anti-cancer drugs or other agents at a tumor site, resulting in markedly reduced toxicity in addition to improved therapeutic activity due to higher drug levels being delivered to the tumor. The use of ionizing radiation in the treatment of cancer has spawned its own medical specialty, radiation oncology. Furthermore, the use of ionizing radiation is a key component of the cancer treatment triad such as surgery, radiation, and chemotherapy. Clinically, the use of ionizing radiation is widespread, and its methods are tremendously sophisticated. Highly defined regions can be irradiated with increasingly homogenous doses. In cancer therapy, radiation and surgery are used primarily in establishing local control of a disease, while chemotherapy is able to reach disseminated disease. However, improved local control alone would have significant effects on clinical outcomes. A significant number of invasive cancers are locally confined at initial diagnosis. Hence, the failure to control a primary tumor results in both increased rates of metastasis and increased numbers of metastatic sites. Thus, improvements in radiation and surgery to provide more effective local control would have a significant impact on clinical outcomes for patients with any of a wide variety of solid tumors. Sterically stabilized liposomes can stay in circulation for extended periods of time and accumulate at tumor sites. Therefore the coupling of localized radiation to trigger the release of agents at these sites provides a significant advantage in local tumor control. Consequently, the use of radiation sensitive liposomes holds tremendous promise in the treatment of disease.

### e) Radiation Sensitive Liposomes as Diagnostics

In another embodiment, the instant invention provides for a method of diagnosing the presence or progression of a disease, comprising the steps of (i) administering to a patient a pharmaceutical composition comprising a liposomal delivery system and releasable agent such as a diagnostic agent, wherein the releasable agent is encapsulated in or associated with the liposome, and a pharmaceutically acceptable carrier or diluent; (ii) subjecting the patient to ionizing radiation in order to destabilize the liposome and release the diagnostic agent encapsulated in or associated with the liposome; and (iii) diagnosing the disease through use of molecular imaging techniques. In one embodiment, the radiation dosage ranges from about 50 to about 2000 rads. In another embodiment, the radiation dosage ranges from about 100 to about 3000 rads. In another embodiment, the radiation dosage ranges from about 500 to about 2500 rads. Examples of diagnostic agents include, but are not limited to, contrast agents, iodinated agents, radiopharmaceuticals, antibodies, fluorescent compounds and fluorescent compounds coencapsulated with a quencher, agents containing MRS/MRI sensitive nuclides, and genetic material encoding contrast agents. Examples of contrast agents include, but are not limited to, gadolinium-diethylenetriaminepentaacetic acid (GdDTPA; Magnavist), aka gadopentetate dimeglumine, gadoteridol (ProHance), gadodiamide, gadoterate meglumine, gadobenate dimeglumine (Gd-BOPTA/Dimeg; MultiHance), mangafodipir trisodium (Mn-DPDP), ferumoxides, paramagnetic analogue of doxorubicin, and ruboxyl (Rb). Examples of iodinated agents include, but are not limited to, diatrizoate (3,5-di(acetamido)-2,4,6-triiodobenzoic acid), iodipamide (3,3′-adipoyl-diimino-di(2,4,6-triiodobenzoic acid), acetrizoate [3-acetylamino-2,4,6-triiodobenzoic acid], aminotrizoate [3-amino-2,4,6-triiodobenzoic acid]), and iomeprol. Examples of radiopharmaceuticals include, but are not limited to, fluorine-18 fluorodeoxyglucose ([¹⁸F]FDG), Tc-99m Depreotide, carbon-11 hydroxyephedrine (HED), [¹⁸F]setoperone, [methyl-¹¹C]thymidine, 99 mTc-hexamethyl propyleneamine oxime (HMPAO), 99 mTc-L, L-ethylcysteinate dimer (ECD), 99 mTc-sestamibi, thallium 201, I-131metaiodobenzylguanidine (MIBG), 123I—N-isopropyl-p-iodoaamphetamine (IMP), 99 mTc-hexakis-2-methoxyisobutylisonitrile (MIBI), 99 mTc-tetrofosmin. Examples of agents containing MRS/MRI sensitive nuclides include, but are not limited to, perfluorocarbons and fluorodeoxyglucose. Examples of genetic material encoding contrast agents include, but are not limited to, paramagnetic reporter genes such as ferredoxin; paramagnetic tag(s) on liposomal lipids such as paramagnetic chelating groups added to PEG; detectable probes; and luciferin/luciferase reporter system. Examples of molecular imaging techniques include, but are not limited to, Nuclear Magnetic Resonance (NMR), Magnetic Resonance Spectroscopy/Magnetic Resonance Imaging (MRS/MRI), X-ray/computed axial tomography (CT), Positron Emission Tomography (PET), Single-photon Emission Computed Tomography (SPECT), ultrasound, and optical based imaging techniques. Examples of conditions that can be diagnosed via liposome-encapsulated or associated diagnostic agents include, but are not limited to, cancer, immune disorders, developmental disorders, and genetic disorders.

The liposomes of the present invention can deliver diagnostic agents to specific sites in the body. For example, the liposomes can selectively localize diagnostic agents at tumor sites, thereby allowing for detection and diagnosis of various malignancies. Liposomes may be targeted to such tumor sites through the attachment of antibodies, antibody fragments, antigens, peptide sequences, peptide fragments, and the like (supra). For example, liposomes with a specific epitope attached may be targeted to a tumor tissue. After binding of the epitope to the receptor of the tumor tissue, the liposomes are irradiated to release their diagnostic contents. Molecular imaging techniques can then be employed to scan and record the diagnostic agents released by the liposomes. This allows for early detection of tumors and other malignancies. Alternatively, radiation sensitive liposomes can be tagged with dyes, fluorescent molecules, radioisotopes, or the like, for purposes of monitoring their progressive travel to tumor sites. Once the liposomes have arrived at a tumor site, the liposomes are irradiated to release their diagnostic contents which can then be used to measure the degree of invasion of the tumor.

### f) Radiation Sensitive Liposomes as a Method of Delivery

In another embodiment, the instant invention provides for a method of delivering releasable agent to site of interest, comprising the steps of (i) administering to a patient a pharmaceutical composition comprising a liposomal delivery system and releasable agent such as a diagnostic agent or a therapeutic agent, wherein the releasable agent is encapsulated in or associated with the liposome, and a pharmaceutically acceptable carrier or diluent; (ii) subjecting the patient to ionizing radiation in order to destabilize the liposome and release the diagnostic agent encapsulated in or associated with the liposome; and (iii) diagnosing the disease through use of molecular imaging techniques. In one embodiment, the radiation dosage ranges from about 50 to about 2000 rads. In another embodiment, the radiation dosage ranges from about 100 to about 3000 rads. In another embodiment, the radiation dosage ranges from about 500 to about 2500 rads. Examples of diagnostic agents include, but are not limited to, contrast agents, iodinated agents, radiopharmaceuticals, antibodies, fluorescent compounds and fluorescent compounds coencapsulated with a quencher, agents containing MRS/MRI sensitive nuclides, and genetic material encoding contrast agents. Examples of contrast agents include, but are not limited to, gadolinium-diethylenetriaminepentaacetic acid (GdDTPA; Magnavist), aka gadopentetate dimeglumine, gadoteridol (ProHance), gadodiamide, gadoterate meglumine, gadobenate dimeglumine (Gd-BOPTA/Dimeg; MultiHance), mangafodipir trisodium (Mn-DPDP), ferumoxides, paramagnetic analogue of doxorubicin, and ruboxyl (Rb). Examples of iodinated agents include, but are not limited to, diatrizoate (3,5-di(acetamido)-2,4,6-triiodobenzoic acid), iodipamide (3,3′-adipoyl-diimino-di(2,4,6-triiodobenzoic acid), acetrizoate [3-acetylamino-2,4,6-triiodobenzoic acid], aminotrizoate [3-amino-2,4,6-triiodobenzoic acid]), and iomeprol. Examples of radiopharmaceuticals include, but are not limited to, fluorine-18 fluorodeoxyglucose ([¹⁸F]FDG), Tc-99m Depreotide, carbon-11 hydroxyephedrine (HED), [¹⁸F]setoperone, [methyl-¹¹C]thymidine, 99 mTc-hexamethyl propyleneamine oxime (HMPAO), 99 mTc-L, L-ethylcysteinate dimer (ECD), 99 mTc-sestamibi, thallium 201, I-131metaiodobenzylguanidine (MIBG), 123I—N-isopropyl-p-iodoamphetamine (IMP), 99 mTc-hexakis-2-methoxyisobutylisonitrile (MIBI), 99 mTc-tetrofosmin. Examples of agents containing MRS/MRI sensitive nuclides include, but are not limited to, perfluorocarbons and fluorodeoxyglucose. Examples of genetic material encoding contrast agents include, but are not limited to, paramagnetic reporter genes such as ferredoxin; paramagnetic tag(s) on liposomal lipids such as paramagnetic chelating groups added to PEG; detectable probes; and luciferin/luciferase reporter system. Examples of molecular imaging techniques include, but are not limited to, Nuclear Magnetic Resonance (NMR), Magnetic Resonance Spectroscopy/Magnetic Resonance Imaging (MRS/MRI), X-ray/computed axial tomography (CT), Positron Emission Tomography (PET), Single-photon Emission Computed Tomography (SPECT), ultrasound, and optical based imaging techniques. Examples of conditions that can be diagnosed via liposome-encapsulated or associated diagnostic agents include, but are not limited to, cancer, immune disorders, developmental disorders, and genetic disorders. Examples of chemotherapeutics include, but are not limited to, alkylating agents such as nitrogen mustards (e.g., chlorambucil, estramustine, mechlorethamine, melphalan); ethylenimine derivatives such as thiotepa; alkyl sulfonates such as busulfan; nitrosoureas such as carmustine (BCNU), lomustine (CCNU), methyl-CCNU, and streptozocin; triazines such as dacarbazine; metal salts such as cisplatin and carboplatin; antimetabolites; folic acid analogues such as methotrexate; pyrimidine analogues such as 5-fluorouracil and floxuridine; purine analogues such as 6-mercaptopurine, 6-thioguanine, deoxycoformycin, and fludarabine; natural products such as vinca alkaloids (e.g., vinblastine and vincristine); podophyllum derivatives such as etoposide and teniposide; antibiotics such as, bleomycin, dactinomycin, doxorubicin, mithramycin, mitomycin, mitoxantrone; hormones and hormone antagonists; androgens such as halotestin, testolactone; corticosteroids such as prednisone and dexamethasone; estrogens such as dethylstilbestrol; progestins megestrol acetate and medroxyprogesterone acetate; estrogen antagonists tamoxifen and taxol; androgen antagonists such as flutamide; LHRH agonists such as leuprolide and goserelin; substituted ureas such as hydroxyurea; methylhydrazine derivatives such as procarbazine; adrenocortical suppressants such as mitotane; steroid synthesis inhibitors such as aminoglutethemide; and substituted melamines such as altretamine. Examples of biological response modifiers include, but are not limited to, interferons such as interferon alpha and interferon gamma; interleukins such as IL-2; and tumor necrosis factor. Examples of radiopharmaceuticals include, but are not limited to, meta-iodobenzylguanidine (MIBG) and ¹¹¹In-labeled somatostatin analog [diethylenetriaminepentaacetic acid (DTPA)-DPhe1]-octreotide. Examples of cell toxins include, but are not limited to, tirapazamine and others. Examples of radiation sensitizers include, but are not limited to, nitroimidazoles such as misonidazole, etanidazole, metronidazole, and nimorazole; docetaxel, paclitaxel, idoxuridine, fludarabine, gemcitabine, and taxanes. Examples of genetic materials include, but are not limited to, chromosome(s), DNA, cDNA, genomic DNA, mRNA, polynucleotide(s), oligonucleotide(s), nucleic acid(s), any synthetic DNA and RNA sequences, and sense and antisense nucleic acid(s). The liposomes of the instant invention can deliver therapeutic agents to specific sites in the body. For example, the liposomes can selectively localize anti-cancer drugs or other agents at a tumor site, resulting in markedly reduced toxicity in addition to improved therapeutic activity due to higher drug levels being delivered to the tumor.

### EXAMPLES

The following specific examples are intended to illustrate the invention and should not be construed as limiting the scope of the claims. The examples further illustrate some of the specifics of destabilization of liposomes and methods employed.

### Example 1

### Radiation Sensitive Liposomes

1.1 Methods

1.1.1 Materials

The polymerizable lipids were synthesized via procedures we have published (Lamparski et al., Biochemistry 1992, 31:685-694; Sells et al., Macromolecules 1994, 27:226-233; Lamparski et al., Macromolecules 1995, 28:1786-1794). Lipid structure was determined by H-NMR, ¹³C-NMR, and mass spectrometry. The purity was examined by thin-layer chromatography with chloroform/methanol/water (65:25:4 by volume) and differential scanning calorimetry (Lamparski et al., J. Am. Chem. Soc. 1993, 115:8096-8102). Pure lipids eluted to a single spot with an R_{f} of 0.35-0.40, and exhibited a sharp highly cooperative main phase transition temperature. Stock benzene solutions of polymerizable lipids (ca. 20 mg/ml) were stored at −40° C. as an amorphous ice. 1,2-Dioleoyl-sn-glycero-3-phosphatidylcholine (DOPC) and distearoyl-sn-glycero-3-phosphatidylcholine (DSPC) were purchased (Avanti Polar Lipids, Inc.) as 20 mg/ml solution in CHCl₃ and stored at −40° C. Buffers and EDTA were purchased (Sigma-Aldrich, Inc.) for buffer preparations and used as received. All buffer solutions were prepared with Milli-Q water (Millipore, Inc.). 8-Aminonaphthalene-1,3,6-trisulfonic acid disodium salt (ANTS) and p-xylenebispyridinium bromide (DPX) were purchased (Molecular Probes, Inc.) as solids and stored at −20° C.

1.1.2 Preparation of Liposomes

The lipids used to prepare the liposomes were each dried under an argon stream followed by drying under vacuum (i.e., vacuum desiccation) for more than two hours and then weighed. This procedure removed the solvent and provided a dry, thin lipid film. Hydration of the lipid film occurred upon addition of aqueous buffer followed by vortex mixing at temperatures greater than the main phase transition temperature to loosen the film from the flask walls. The lipids were hydrated in the desired molar ratio, including the agents to be encapsulated. The hydrated bilayers were converted into liposomes through the following procedure: Ten freeze/thaw cycles consisting of freezing by submersion of the solution vial into −78° C. dry ice/isopropanol bath followed by thawing in a 37° C. bath, insuring the lipids were well hydrated and were in extended bilayers. The lipid bilayers were converted to liposomes of defined size by extrusion at 37° C. through 2 stacked 100 nm pore size Nuclepore brand polycarbonate filters through a stainless extrusion apparatus (Lipex Biomembranes, Inc.). The liposomes were purified by elution through a gel permeation chromatography column with an isoosmotic buffer to remove unencapsulated agents. The liposome size distributions were measured by dynamic light scattering. Multiangle dynamic laser light scattering provided vesicle diameter distributions upon analysis of data obtained from 90°-, 60°- and 1200 angle using various fitting methods, i.e., single exponential sampling, non-negatively constrained least squares and CONTIN as previously published (Koelchens et al., Chem. Phys. Lipids 1993, 65:1-10). Liposomes were 100±10 nm in diameter.

1.2 Results

The relative sensitivity to ionizing radiation of liposomes composed of either bis-SorbPC (1; FIG. 5), bis-DenPC (2; FIG. 5), or bis-AcrylPC (3; FIG. 5) was investigated. These polymerizable colipids have different reactivities that control the initial rate of polymerization, extent of polymerization, and inhibition by oxygen. The interaction of ionizing radiation with water produces radical species that can initiate radical chain polymerizations. Radical polymerization of acryloyl monomers generally occurs at a faster rate than diene containing monomers, such as the dienoyl and sorbyl esters, because the propagating radicals are less stable (Odian, G., Principles of Polymerization, 1991, John Wiley & Sons, New York).

The rate of the ionizing radiation initiated polymerization (Rp) of liposomes composed of either bis-SorbPC, bis-DenPC, or bis-AcrylPC was determined by the loss of monomer absorption intensity. The maximum dose rate at the liposome samples was 2.1×10⁴ rad/h. This intensity could be attenuated if desired. The ionizing radiation initiated polymerization of bis-SorbPC and bis-DenPC was relatively insensitive to the presence of dissolved oxygen in the aqueous buffer, whereas the polymerization of bis-AcrylPC was inhibited by oxygen. There was no change in the monomer concentration even after 120 minutes of irradiation at the maximum dose rate. Radical polymerizations of acryloyl functionalities are very sensitive to oxygen due to the high energy of the propagating species. In contrast, if the bis-AcrylPC liposome suspension was purged of oxygen, the monomer could be converted to polymer in a few minutes. The Rp for bis-SorbPC liposomes in water (with or without oxygen present) was about twice that of bis-DenPC liposomes. The liposomes composed of bis-AcrylPC (without oxygen) were about 10 times more reactive than bis-SorbPC liposomes. These results are similar to those obtained for the polymerization of these monomers via the generation of radicals from thermal initiators.

We showed that the extent of polymerization of bis-SorbPC and bis-AcrylPC was independent of whether the exposure to ionizing radiation was continuous or discontinuous. The usual experimental protocol for radiation exposure of the liposome samples utilized discontinuous exposure because samples for all the time points were exposed together. Hence, the source was on to expose the samples and switched off to permit sample retrieval at each time point. In some experiments the samples were exposed continuously. The percent loss of monomer depended on the total exposure, but was insensitive to whether the radiation was delivered in a continuous or punctuated manner.

### Example 2

### Ionizing Radiation as a Trigger for PEG-Liposome Destabilization

In order to determine whether relatively low doses of ionizing radiation, i.e., comparable to therapeutic doses, could be effective in destabilizing PEG-liposomes, liposomes were prepared with encapsulated water soluble fluorescent markers. The release of these markers was then determined as a function of the dose of ionizing radiation. The following experiments demonstrate that doses as low as 50 rads can cause the release of water soluble markers from PEG-liposomes.

2.1 Methods

2.1.1 Liposome Preparation

The polymerizable lipids used, and the preparation of the liposomes are all as described in Section 1 (supra).

2.1.2 Liposomal Irradiation

After preparation and purification via column chromatography, liposomes coencapsulating ANTS and its collisional quencher DPX were irradiated using a Cobalt-60 teletherapy unit (Arizona Cancer Center Experimental Radiation Facility). Irradiation was carried out at doses ranging from 0 to 1000 rads. Leakage was assessed at time points ranging from 0 to 36 hours.

2.1.3 Liposome Destabilization

The radiation induced release of liposomal contents was monitored by dequenching of ANTS fluorescence as ANTS and DPX (collisional quencher of ANTS fluorescence) were released and diluted into the bulk aqueous phase (Bennett et al., Biochemistry 1995, 34:3102-3113; Ellens et al., Biochemistry 1985, 24:3099-3106). The liposome population coencapsulated ANTS and DPX by preparing them in a buffer consisting of 25 mM ANTS with 90 mM DPX and 10 mM phosphate pH 7.5 287 miliosmole.

Untrapped ANTS and DPX were separated from the liposomes by passing the suspension over a SEC column packed with Sephadex G50 or G75 gel. Leakage was monitored by fluorescence dequenching vs. time with excitation at 360 nm and emission observed at 520 nm. The sample cell holder was thermostatted to the appropriate temperature and the sample was continuously stirred. The 100% leakage value was obtained by lysing the liposomes with excess Triton X-100. Time based fluorescence scans were converted to % leakage vs. time (seconds) plots using the following equation:

100×(Iₜ−bIₒ)/(Iₜᵣᵢₜₒₙ−bIₒ)

Iₜ refers to the fluorescence intensity at some time t; Iₒ is the initial fluorescence value; Iₜᵣᵢₜₒₙ is the relative fluorescence after 100% leakage due to triton lysis of vesicles corrected for change in concentration; and b is a bleaching factor to correct for any bleaching due to ionizing irradiation of the fluorescent dye, ANTS, during the experiment.

2.2 Results

In order to assess the effect of ionizing radiation on polymerizable PEG-liposomes, various liposome compositions were prepared. The lipid ratios used for preparation of the PEG-liposomes compositions were the following:

Composition 1: PEG₂₀₀₀-dioleoylPE, cholesterol, dioleoylPC, and bis-SorbPC_{17,17} (molar ratio: 15/40/20/30)

Composition 2: PEG₂₀₀₀-distearoylPE, cholesterol, distearoylPC, and bis-SorbPC_{17,17} (molar ratio: 15/40/20/30)

For liposomes prepared from Composition 1, significant release was observed at doses as low as 250 rads. Increasing release was observed for a given dose with increasing time. Increasing release was also observed with increasing doses up through 2500 rads. For liposomes prepared from Composition 2, significant release was observed at doses as low as 50 rads. Increasing release was seen with increasing doses up to 200-250 rads. Radiation doses higher than 250 rads show little or no significant increases in release. At all doses examined, no significant increases in release were observed over time.

Additional PEG-liposome compositions used to test the release of liposomal contents were the following:

Composition 3: PEG₂₀₀₀-distearoylPE, distearoylPC, and bis-SorbPC_{17,17} (molar ratio 5/75/20)

Composition 4: PEG₂₀₀₀-dioleoylPE, cholesterol, dioleoylPC, and bis-SorbPC₁₇₋₁₇ (molar ratio 15/35/18/31)

Composition 5: PEG₂₀₀₀-distearoylPE, cholesterol, distearoylPC, and bis-SorbPC_{17,17} (molar ratio 16/35/20/28)

Composition 6: PEG₂₀₀₀-distearoylPE, cholesterol, distearoylPC, and bis-SorbPC_{17,17} (molar ratio 15/35/20/30)

Composition 7: PEG₂₀₀₀-distearoylPE, cholesterol, distearoylPC, and bis-SorbPC_{17,17} (molar ratio 15/34/21/30)

Given that clinical doses in current cancer therapy are typically 2 Gy (200 rads), these experiments clearly demonstrate the potential clinical utility of radiation-induced liposomal destabilization for the treatment of cancer. They also suggest that adjusting the exact lipid composition of the liposome can alter the release to meet specific needs, i.e., significant release with minimal irradiation or increasing release over time after irradiation. The data further suggests that significant release can be achieved at subclinical doses, suggesting the potential use of ionizing radiation as a trigger for liposomal release for the treatment of other non-cancerous conditions.

### Example 3

### Ionizing Radiation Induced Release of Doxorubicin

In order to further investigate the release of encapsulated agents from the radiation sensitive liposomes, we conducted additional experiments that measured doxorubicin release. Our experiments indicated that 100-200 rads were required to cause significant leakage of encapsulated doxorubicin from the PEG-liposomes. Thus, as the following experiments demonstrate, we have shown that doses even as low as 100 to 200 rads can cause the release of encapsulated agents from PEG-liposomes.

3.1 Methods

3.1.1 Liposome Preparation

The lipid ratios used for preparation of the PEG-liposomes composition used in this experiment were the following:

Composition 8: PEG₂₀₀₀-distearoylPE, cholesterol, distearoylPC, and bis-SorbPC_{17,17} (molar ratio Apr. 34, 1942/20).

The liposomes were hydrated with 3 mL of 120 mM (NH₄)₂SO₄ (in MilliQ water, pH 7.0). The sample was freeze/thawed ten times at dry ice/isopropanol and 60° C. The sample was then extruded twice through a series of decreasing size Nuclepore filters with the smallest pores being 100 nm in diameter. The liposomes were stored in a refrigerator overnight. The next day the ammonium sulfate was removed from the outside of the liposomes by exchange with aqueous NaCl (pH 7.0, 270 mOsm=isoosmotic) on a Sephadex G-75 column. The total lipid concentration of the fraction from the column was 2.77 mM.

Doxorubicin (0.5 mM, 10 mg/ml in MilliQ water) was added and incubated with the liposome sample for 15 minutes at 30° C. The sample was kept on ice for a few minutes and was then equilibrated for 5 minutes, before the next column was run. This Sephadex G-75 column exchanged the doxorubicin from the outside of the PEG-liposomes with aqueous NaCl (pH 7.0, 270 mOsm=isoosmotic). The collected liposomes were used to determine their sensitivity to ionizing radiation.

3.2 Results

The radiation induced release of doxorubicin was determined (in triplicate) by the increased fluorescence of each sample. An exposure of 100 rads resulted in the release of 50% of the doxorubicin. When the exposure was 200 rads, 90% of the doxorubicin was released, thus demonstrating that doses as low as 100 to 200 rads can cause the release of encapsulated therapeutic agents from radiation sensitive liposomes.

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the claims.

### Example 4

### Preparation of Thiol Chain Transfer Agents

### Synthesis for the Product (10E,12E)-4,7-dimethylenetradecca-10,12-diene-1-thiol. (See FIG. 6)

A Grignard reaction of 4-tertbutyldiphenylsilyl-oxy-butyraldehyde (I) with 3-bromopropionaldehyde-ethylene glycol acetal (II) gives the differentially protected secondary alcohol (III). This hydroxyl is then converted to the benzyl ether under basic conditions, and deprotected at the aldehyde functionality with trifluoroacetic acid to yield the aldehyde (IV). This is then reacted with another equivalent of the Grignard reagent (II) to yield the decane tri-ether-acetal (V). Removal of the benzyl protecting groups by catalytic hydrogenation followed by oxidation of the resulting hydroxyls with pyridinium dichromate gives the decane-dione (VI). Reaction with two equivalents of methylene Wittig reagent under basic conditions gives the dimethylene compound (VII). This is then deprotected and a second Wittig reaction with 2-E-propenyl triphenylphosphoniumbromide gives the dimethylene diene ether (X). This is then deprotected with tributylammonium fluoride and converted to an activated tosylate ester, which is converted to the corresponding thiol (XI) with thiourea followed by potassium hydroxide in methanol.

### Synthesis for the Product (4E,6E,11E,13E)-pentadeca-4,6,11,13-tetraene-1-thiol (FIG. 7)

The tetrahydropyranyl-protected 7-bromoheptanol is converted to the corresponding triphenylphosphonium salt (II). This is used in a Wittig reaction with propionaldehyde to produce the alkene (III). Acid deprotection followed by a Swem oxidation produces the aldehyde (4). Wittig reaction with the triphenyl phosphonium salt (VI) produces the diene (VII). This is brominated and then converted to the bis-diene (VIII) with base. Deprotection followed by tosylation produces the activated ester (IX). This is then converted to the thiol (X) by reacting with thiourea followed by methanolic potassium hydroxide.

### Example 5

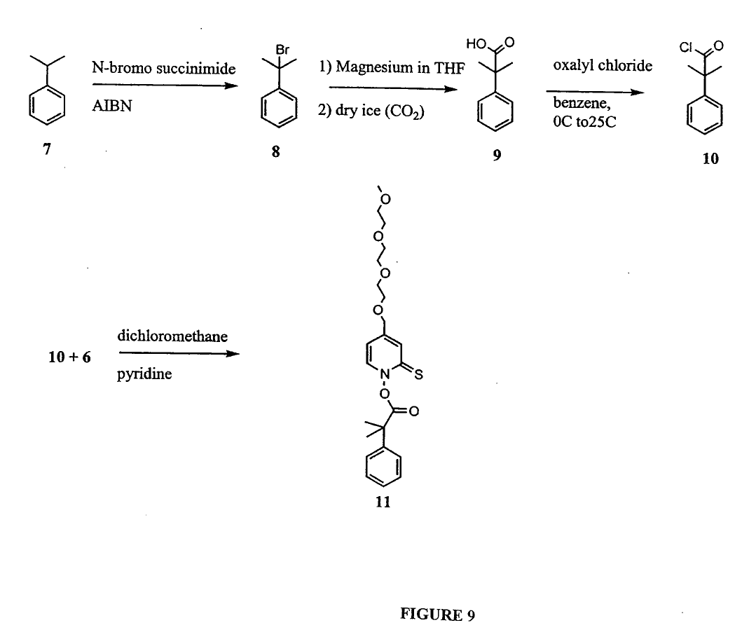

### Preparation of Barton Ester Chain Transfer Agents (FIGS. 8 and 9)

### Synthesis of 4-(bromomethyl)-pyridine-1-oxide (Compound 2 in FIG. 8)

4-methyl-pyridine-1-oxide, (1 eq), N-bromo-succinimide (1.2 eq.), and AIBN are combined in benzene. A reflux condenser is fitted to the flask, and the system is purged with argon. The solution is brought to reflux with magnetic stirring and allowed to reflux for 8 hr. Succinimide is removed by vacuum filtration and the solvent is evaporated under reduced pressure. The product is purified by crystallization from the appropriate solvent or by chromatography in a slightly acidic medium. [March, J., Advanced Organic Chemistry. 4^{th} ed. 1992, New York, N.Y.: John Wiley and Sons]

### Synthesis of 4-(1-ethoxy-2-ethoxy-2-ethoxy-2-methoxy-methyl)-pyridine-1-oxide (Compound 3 in FIG. 8)

Mono-methoxy triethyleneglycol (1.2 eq) is dissolved in dry THF. Potassium hydride (1.4 eq) is added, and the solution is stirred. The solution will turn purple. 4-(bromomethyl)-pyridine-1-oxide (2) (1 eq) is added, and the reaction is stirred at room temperature under argon for 3 hours. Water (1 eq) and HCl (0.5 eq) are added. The solvent is removed under reduced pressure, and the product is purified by chromatography on silica gel with ethyl acetate as a solvent [March, J., Advanced Organic Chemistry. 4^{th} ed. 1992, New York, N.Y.: John Wiley and Sons]

### Synthesis of 4-(1-ethoxy-2-ethoxy-2-ethoxy-2-methoxy-methyl)-2-bromo-pyridine-1-oxide (Compound 4 in FIG. 8)

4-(1-ethoxy-2-ethoxy-2-ethoxy-2-methoxy-methyl)-pyridine-1-oxide is dissolved in dry THF and cooled to −65° C. Butyl lithium (1 eq) is added and the reaction is stirred for 10 minutes. Bromine (1 eq) is then added and the solution is allowed to warm to room temperature. The solution is filtered and the filtrate is evaporated at reduced pressure. The product is separated by chromatography on silica gel. [Abramovitch, R. A., et al., Journal of Heterocyclic Chemistry, 1972. 9: p. 13672]

### Synthesis of 4-(1-ethoxy-2-ethoxy-2-ethoxy-2-methoxy-methyl)-2-isothiourea-pyridine-1-oxide hydrobromide (Compound 5 in FIG. 8)

4-(1-ethoxy-2-ethoxy-2-ethoxy-2-methoxy-methyl)-2-bromo-pyridine-1-oxide (1 eq.) and thiourea (1 eq) is dissolved in ethyl alcohol and brought to reflux for 1 hour. The product is separated by precipitation and vacuum filtration. [Shaw, E., et al., Analogs of Aspergillic Acid. IV. Substituted 2-Bromopyridine-N-oxides and Their Conversion to Cyclic Thiohydroxamic Acids. Journal of the American Chemical Society, 1950. 72: p. 4362-4364]

### Synthesis of 4-(1-ethoxy-2-ethoxy-2-ethoxy-2-methoxy-methyl)-1-hydroxy-pyridine-2-thione (compound 6 in FIG. 8)

4-(1-ethoxy-2-ethoxy-2-ethoxy-2-methoxy-methyl)-2-isothiourea-pyridine-1-oxide hydrobromide (1 eq) is dissolved in water with sodium carbonate (1.9 eq) and allowed to stand at 25° C. for 4 hours. The solution is then acidified. The product is collected by filtration or by extraction into chloroform. [Shaw, E., et al., Analogs of Aspergillic Acid. IV

Substituted 2-Bromopyridine-N-oxides and Their Conversion to Cyclic Thiohydroxamic Acids. Journal of the American Chemical Society, 1950. 72: p. 4362-4364]

### Synthesis of α-Bromo Cumene (Compound 8 in FIG. 9)

Cumene (1 eq), N-bromo succinimide (1 eq) and AIBN (0.1 eq) are combined in benzene. The solution is purged under argon, and brought to reflux. The reaction is allowed to run at reflux under argon for 8 hours. The solvent is then evaporated under reduced pressure, and the product is purified by vacuum distillation. [March, J., Advanced Organic Chemistry. 4^{th} ed. 1992, New York, N.Y.: John Wiley and Sons]

### Synthesis of 2-methyl-2-phenyl propanoic acid (Compound 9 in FIG. 9)

Finely divided Magnesium (1.5 eq) is suspended in dry THF under argon. Iodine (0.05 eq) followed by α-bromo cumene are added slowly while the solution is heated gently until a vigorous reaction occurs. The solution is cooled, and excess dry ice is added. Solid inorganics are separated by vacuum filtration. The solvent is evaporated and the residue is dissolved in chloroform, and washed with 1 N hydrochloric acid. The product is purified by crystallization from the appropriate solvent. [March, J., Advanced Organic Chemistry. 4^{th} ed. 1992, New York, N.Y.: John Wiley and Sons]

### Synthesis of 2-methyl-2-phenyl propanoyl chloride (Compound 10 in FIG. 9)

2-methyl-2-phenyl propanoic acid is dissolved in benzene under argon and cooled to 1° C. Oxalyl chloride (1.2 eq) is added, and the solution is allowed to stir for 4 hr while it warms gradually to room temperature. The solvent is evaporated under reduced pressure with a KOH trap, and the product is purified by vacuum distillation. [March, J., Advanced Organic Chemistry. 4^{th} ed. 1992, New York, N.Y.: John Wiley and Sons]

### Synthesis of 2-methyl-2-phenylpropionoyl-4-(1-ethoxy-2-ethoxy-2-ethoxy-2-methoxy-methyl)-1-hydroxy-pyridine-2-thione (Compound 11 in FIG. 9)

4-(1-ethoxy-2-ethoxy-2-ethoxy-2-methoxy-methyl)-1-hydroxy-pyridine-2-thione (1 eq) and pyridine (2 eq) are combined in dry dichloromethane under argon. The solution is cooled to 0° C. and 2-methyl-2-phenyl propanoyl chloride (1 eq) is added. The solution is allowed to reach room temperature and stirred for 4 hours under argon. The reaction mixture is filtered and the solvent is evaporated from the filtrate under reduced pressure. The product is purified by solid phase extraction from a cation exchange resin followed by chromatography. [Sells, T. D. and O'Brien, D. F., Two-Dimensional Polymerization of Lipid Bilayers: Degree of Polymerization of Acryloyl Lipids. Macromolecules, 1994. 27: p. 226-233].

### Example 6

### Preparation and Polymerization of Liposomes with Chain Transfer Agents

Lipid totaling 0.02 millimoles and having a composition of bisSorbPC_{17,17} (65 mole %), cholesterol (30 mole %) and PEG₂₀₀₀-DSPE (5 mole %) were combined, one component at a time, in chloroform and dried each time under a stream of nitrogen gas. Each successive addition was dried under 0.1 mm Hg for 8 hr and weighed to obtain an accurate composition. The lipid mixture was dissolved in 1.00 ml of chloroform, and divided into four equal 5250 μL parts in four different 15 ml round bottom flasks. Each was evaporated and dried as above. Chloroform (400 μl) was added to each flask. 0.01 millimole of octanethiol (here designated as octanethiol, or O) was added to two of the four flasks. No additional component was added to the other flask (here designated as control or C). The samples were then evaporated and dried as previously described.

Dulbecco's 1×PBS was added to one of the dried C sample and one of the dried O sample, respectively, and Dulbecco's 1×PBS with 15 μM glutathione was added to the other dried C sample and the other dried 0 sample, respectively. All four samples were purged with argon and freeze-thawed 10 times under argon with a high temperature of 60° C. and low temperature of −78° C., resulting in liposomes. The liposomes in each 15 ml flask were then extruded, at 60° C., 2 times through 2 stacked 200 nm polycarbonate membranes followed by 10 times through two stacked 100 nm polycarbonate membranes. The samples were allowed to sit under argon, at room temperature over night. The next day, the concentration of each sample was measured by the UV absorbance at 250 nm of 30 μL liposome solution in 3 ml HPLC grade methanol. The concentration for C was found to be 4.2 mM and for 0 was found to be 4.7 mM.

According to these concentrations, 4 solutions of 75 micromolar total lipid were made, these are: C in Dulbecco's PBS, C with 15 micromolar glutathione in Dulbecco's PBS; O in Dulbecco's PBS, and O with 15 micromolar glutathione in Dulbecco's PBS. The UV absorbance spectrum of each of these solutions was measured from 200 nm to 400 nm. Samples of each solution were exposed, at 37° C., in duplicate (a and b), to 0, 20, and 50 Gy radiation from a 137Cs source at 6.74Gy/min. The UV spectrum of each sample was measured and the % polymerization was calculated. Error bars represent the square root of the square of the difference in the y values at each point.

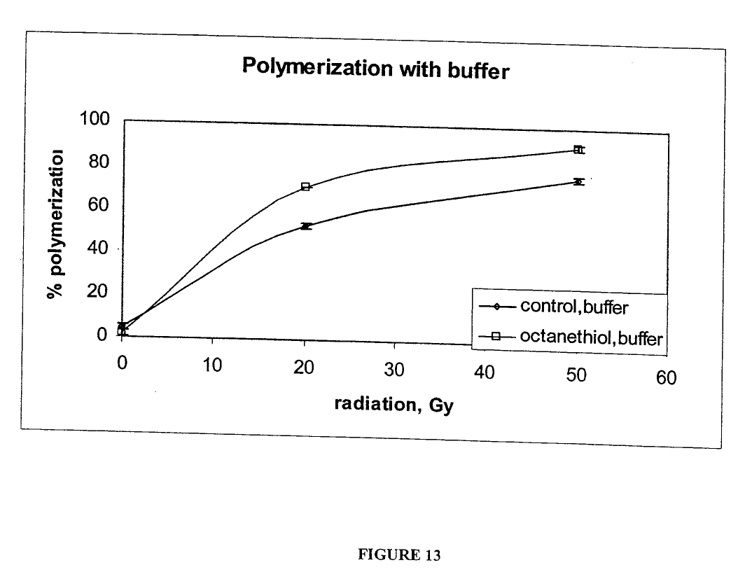

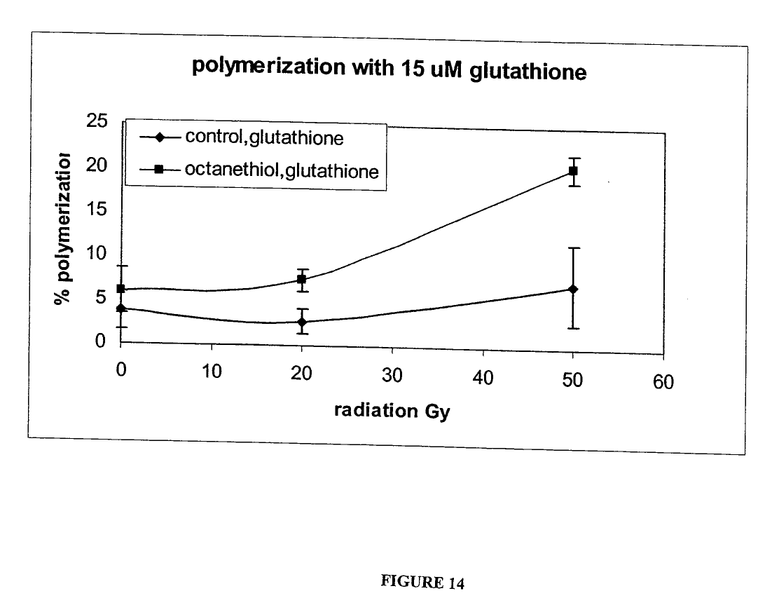

The data shown below show a significant re-sensitization of the liposomes with octanethiol in 15 μM glutathione (FIG. 13), as well as a significant increase in the rate of polymerization with octanethiol in PBS buffer (FIG. 14).

What is claimed is:

1. A polymerizable liposome, comprising a stable liposome-forming lipid, a radiation polymerizable colipid, a chain transfer agent and a releaseable agent.

2. The liposome of claim 1 wherein the chain transfer agent has a cleavable hydrophilic group which is cleaved upon exposure to radiation.

3. The liposome of claim 1 wherein the chain transfer agent is located near the liposome surface.

4. The liposome of claim 1 wherein the chain transfer agent is an unsaturated thiol.

5. The liposome of claim 1 wherein the chain transfer agent is selected from the group consisting of amphiphilic halocarbon chain transfer agents, amphiphilic tertiary amine chain transfer agents, thiol chain transfer agents, unsaturated thiol chain transfer agents, branched thiol chain transfer agents, Barton esters, and thiol cholesterols.

6. The liposome of claim 1 further comprising a steric stabilizer.

7. The liposome of claim 6 wherein the steric stabilizer is polyethylene glycol functionally attached to a lipid.

8. The liposome of claim 1 further comprising at least one peptide.

9. The liposome of claim 8 wherein the peptide is selected from the group consisting of peptide sequences, peptide fragments, antibodies, antibody fragments and antigens.

10. The liposome of claim 1 wherein the lipid is selected from the group consisting of phosphatidylcholine (PC), phosphatidylethanolamine (PE), phosphatidic acid (PA), phosphatidylglycerol (PG), nonnatural lipid(s), and cationic lipid(s).

11. The liposome of claim 1 wherein the radiation polymerizable colipid is selected from the group consisting of mono-lipids with polymerizable moieties, bis-lipids with polymerizable moieties, and mixtures of mono- and bis-lipids with polymerizable moieties.

12. The liposome of claim 1 wherein the polymerizable colipid is selected from the group consisting of mono-, bis-, and heterobifunctional, diacetylenyl, acryloyl, methacryloyl, dienoyl, dienyl, sorbyl, muconyl, styryl, vinyl, and lipoyl colipid(s).

13. The liposome of claim 1 wherein the releasable agent(s) are liposome-encapsulated or associated molecules.

14. The liposome of claim 13 wherein the releasable agent(s) are selected from the group consisting of therapeutic agents and diagnostic agents.

15. The liposome of claim 14 wherein the therapeutic agent is selected from the group consisting of chemotherapeutics, biological response modifiers, biological cofactors, pharmaceuticals and radiopharmaceuticals, cell toxins, radiation sensitizers, and genetic materials.

16. The liposome of claim 14 wherein the diagnostic agent is selected from the group consisting of contrast agents, iodinated agents, radiopharmaceuticals, fluorescent compounds and fluorescent compounds coencapsulated with a quencher, agents containing MRS/MRI sensitive nuclides, and genetic material encoding contrast agents.

17. A polymerizable liposome comprising a stable liposome-forming lipid, a radiation polymerizable colipid and a biologically acceptable oxidizing agent.

18. The liposome of claim 17 further comprising a steric stabilizer.

19. The liposome of claim 17 further comprising at least one peptide.

20. The liposome of claim 19 wherein the peptide is selected from the group consisting of peptide sequences, peptide fragments, antibodies, antibody fragments and antigens.

21. The liposome of claim 17, wherein the lipid is selected from the group consisting of phosphatidylcholine (PC), phosphatidylethanolamine (PE), phosphatidic acid (PA), phosphatidylglycerol (PG), nonnatural lipid(s), and cationic lipid(s).

22. The liposome of claim 17, wherein the radiation polymerizable colipid is selected from the group consisting of mono-lipids with polymerizable moieties, bis-lipids with polymerizable moieties, and mixtures of mono- and bis-lipids with polymerizable moieties.

23. A liposome composition comprising a mixture of a first liposome and a second liposome wherein the first liposome comprises a stable liposome-forming lipid, a radiation polymerizable colipid and a biologically acceptable oxidizing agent and the second liposome comprises a stable liposome-forming lipid, a radiation polymerizable colipid and a releaseable agent.

24. The liposome composition of claim 23, wherein the second liposome may further comprise a chain transfer agent.

25. A pharmaceutical composition comprising (1) the liposome of claim 1 and (2) a pharmaceutically acceptable carrier.

26. A pharmaceutical composition comprising (1) the liposome of claim 17, (2) a liposome comprising a stable liposome forming lipid, (3) a radiation polymerizable colipid, (4) a releaseable agent, and (5) a pharmaceutically acceptable carrier.

27. A pharmaceutical composition comprising (1) the liposome of claim 1, (2) a liposome comprising a stable liposome forming lipid, (3) a radiation polymerizable colipid, (4) a biologically acceptable oxidizing agent, and (5) a pharmaceutically acceptable carrier.

28. A method of treating a condition responsive to a therapeutic agent, comprising the steps of:
administering to a patient a pharmaceutical composition selected from a group consisting of the pharmaceutical composition of claim 23, the pharmaceutical composition of claim 24, the pharmaceutical composition of claim 25, the pharmaceutical composition of claim 26, and the pharmaceutical composition of claim 27, wherein the releaseable agent is a therapeutic agent; and
subjecting the patient to radiation in order to destabilize the liposome and release the therapeutic agent encapsulated in or associated with the liposome.

29. A method of diagnosing the presence or progression of a disease, comprising the steps of:
administering to a patient a pharmaceutical composition selected from a group consisting of the pharmaceutical composition of claim 23, the pharmaceutical composition of claim 24, the pharmaceutical composition of claim 25, the pharmaceutical composition of claim 26, and the pharmaceutical composition of claim 27, wherein the releasable agent is a diagnostic agent;
subjecting the patient to radiation in order to destabilize the liposome and release the therapeutic agent encapsulated in or associated with the liposome; and
diagnosing the disease through use of molecular imaging techniques.

30. A method of delivering a releasable agent to an area of interest, comprising the steps of:
administering to a patient a pharmaceutical composition selected from a group consisting of the pharmaceutical composition of claim 23, the pharmaceutical composition of claim 24, the pharmaceutical composition of claim 25, the pharmaceutical composition of claim 26, and the pharmaceutical composition of claim 27; and
subjecting the patient to radiation in order to destabilize the liposome and release the releasable agent encapsulated in or associated with the liposome.

31. A method of producing a liposome comprising
mixing a stable liposome-forming lipid, a radiation polymerizable colipid and a chain transfer agent;
hydrating the mixture with a buffer comprising agents to be encapsulated or associated in a desired molar ratio to create hydrated bilayers;
converting the bilayers into liposome(s); and
purifying the liposome(s).

32. A method of producing a liposome comprising
mixing a stable liposome-forming lipid and a radiation polymerizable colipid and hydrating the lipids with a buffer comprising oxidizing agents to be encapsulated or associated in a desired molar ratio to create hydrated bilayers;
converting the bilayers into liposome(s); and
purifying the liposome(s).
